SEQUENCE LISTING (54 sequences)

SEQ ID NO: 1 - RNA, 155 nt, synthetic construct
  source 1..155: mol_type = other RNA
gtgctcgactagggataacagggtaattgtttgaatgaggcttcagtactttacagaatcgttgcctgcacatcttggaaacacttgctgggattacttcagctctttaacccaacagaaggctcgagagaaagcatatctgttgacagtgagcg

SEQ ID NO: 2 - RNA, 177 nt, synthetic construct
  source 1..177: mol_type = other RNA
gtgctcgactagggataacagggtaattgtttgaatgaggcttcagtactttacagaatcgttgcctgcacatcttggaaacacttgctgggattacttcagctctttaacccaacagaaggctcgaggctagcgccgatataacggcggagaaagcatatctgttgacagtgagcg

SEQ ID NO: 3 - RNA, 155 nt, synthetic construct
  source 1..155: mol_type = other RNA
gtgctcgactagggataacagggtaattgtttgaatgaggcttcagtactttacagaatcgttgcctgcacatcttggaaacacttgctgggattacttcacctgtttaacccaacagaaggcttaagagaaagcatatctgttgacagtgagcg

SEQ ID NO: 4 - RNA, 155 nt, synthetic construct
  source 1..155: mol_type = other RNA
gtgctcgactagggataacagggtaattgtttgaatgaggcttcagtactttacagaatcgttgcctgcacatcttggaaacacttgctgggattacttcgacttttctaaccaacagaaggcgtacgagaaagcatatctgttgacagtgagcg

SEQ ID NO: 5 - RNA, 155 nt, synthetic construct
  source 1..155: mol_type = other RNA
gtgctcgactagggataacagggtaattgtttgaatgaggcttcagtactttacagaatcgttgcctgcacatcttggaaacacttgctgggattacttcgacttcttaacccaacagaaggctcgagaaggtatattgctgttgacagtgagcg

SEQ ID NO: 6 - RNA, 60 nt, synthetic construct
  source 1..60: mol_type = other RNA
ctcgagccggatcaacgccctaggtttatgtttggatgaactgacatccgcgtatccgtc

SEQ ID NO: 7 - RNA, 32 nt, synthetic construct
  source 1..32: mol_type = other RNA
ctcgagcagccagctttttgcgaatctcgaca

SEQ ID NO: 8 - RNA, 19 nt, synthetic construct
  source 1..19: mol_type = other RNA
tagtgaagccacagatgta

SEQ ID NO: 9 - RNA, 21 nt, synthetic construct
  source 1..21: mol_type = other RNA
gtagtgaaatatatattaaac

SEQ ID NO: 10 - RNA, 15 nt, synthetic construct
  source 1..15: mol_type = other RNA
tgtgttttttttgaa

SEQ ID NO: 11 - RNA, 132 nt, synthetic construct
  source 1..132: mol_type = other RNA
tgcctactgcctcggacttcaaggggctaagatctggcaattatcttgtttactaaaactgaataccttgctatctctttgatacatttttacaaagctgaattaaaatggtataaattaaatcactttttt

SEQ ID NO: 12 - RNA, 132 nt, synthetic construct
  source 1..132: mol_type = other RNA
tgcctactgcctcggacttcaagggactagtagatctcaattatcttgtttactaaaactgaataccttgctatctctttgatacatttttacaaagctgaattaaaatggtataaattaaatcactttttt

SEQ ID NO: 13 - RNA, 132 nt, synthetic construct
  source 1..132: mol_type = other RNA
tgcctactgcctcggacttcaaggggctaaccggtggcaattatcttgtttactaaaactgaataccttgctatctctttgatacatttttacaaagctgaattaaaatggtataaattaaatcactttttt

SEQ ID NO: 14 - RNA, 131 nt, synthetic construct
  source 1..131: mol_type = other RNA
tgcctactgcctcggacttcaaggggctattcgaagcaattatcttgtttactaaaactgaataccttgctatctctttgatacatttttacaaagctgaattaaaatggtataaattaaatcactttttt

SEQ ID NO: 15 - RNA, 132 nt, synthetic construct
  source 1..132: mol_type = other RNA
tgcctactgcctcggacttcaaggggctagaattcggcaattatcttgtttactaaaactgaataccttgctatctctttgatacatttttacaaagctgaattaaaatggtataaattaaatcactttttt

SEQ ID NO: 16 - RNA, 54 nt, synthetic construct
  source 1..54: mol_type = other RNA
tacggtaacgcggaatacgcaactattttatcaattttttgcgtcgacagactc

SEQ ID NO: 17 - RNA, 30 nt, synthetic construct
  source 1..30: mol_type = other RNA
tcgcgattcgctttttcgtctttgagatct

SEQ ID NO: 18 - RNA, 56 nt, synthetic construct
  source 1..56: mol_type = other RNA
ggaacggtgcattggaacgcggattccccgtgccaagagtgacgtaagtaccgcct

SEQ ID NO: 19 - RNA, 155 nt, synthetic construct
  source 1..155: mol_type = other RNA
atagagtctataggcccacccccttggcttcgttagaacgcggctacaattaatacataaccttatgtatcatacacatacgatttaggtgacactatagaataacatccactttgcctttctctccacaggtgtccactcccaggtccaactgc

SEQ ID NO: 20 - RNA, 18 nt, synthetic construct
  source 1..18: mol_type = other RNA
cagttcgaagaggtaagt

SEQ ID NO: 21 - RNA, 42 nt, synthetic construct
  source 1..42: mol_type = other RNA
tactaactcttctttttttttttcacaggaccatcgatcgaa

SEQ ID NO: 22 - RNA, 18 nt, synthetic construct
  source 1..18: mol_type = other RNA
ccgccgatataacggcgg

SEQ ID NO: 23 - DNA, 890 bp, Cricetulus griseus
  source 1..890: mol_type = other DNA
cttccacttttcctcactcactgacgatgctgcccctggggaccaggctcccagcctgactccacccactggctcactataaaggctgaggcagagctgccagaagagcagagagactgaggccaacccagacctgcctattctctccgaagccagctctctcttcttccaccatgcagttctccgcaagaacgcttctgtgcctgctactcacagttgctgcctgtagcatctatgtgctggcccagccagacgctgttaactcccccctcacctgctgctactcatttaccgccaagaggatcccagagaagaggctggagagctacaagagaatcaccagcagcaagtgtcccaaagaagctgtgattttcatcaccaagctcaagagagagatctgtgctgaccccaagcaggactgggtccagacatacacaaaaaaactggaccagagccaagctaaatcagaagctgcaactgtgtataaaacagcacctttgaatgctaacttgacccatgagtctgcagttaatgcatccactactgcctttcccaccacagacttgagaacttctgtaagagtgaccagtatgacagtgaactagtgtgacctggaatgtgatgccttaagtaatattaactttatttaacttattaatgtttcaatatccccttccctgtatacttggatttcttagatgcatgacgtggtttcattgttttccctctgtgaaccctggttctccaccctaaatgttatgagagatgacttttgtaaagatgagtgaaaaaaaaaacgttgtgtttgagatcccaaggcattgtttaaaatattattatggaaatggatatttctaattattaagaggaataaattatttttgtacata

SEQ ID NO: 24 - protein, 143 aa, Cricetulus griseus
MQFSARTLLCLLLTVAACSIYVLAQPDAVNSPLTCCYSFTAKRIPEKRLESYKRITSSKCPKEAVIFITKLKREICADPKQDWVQTYTKKLDQSQAKSEAATVYKTAPLNANLTHESAVNASTTAFPTTDLRTSVRVTSMTVN

SEQ ID NO: 25 - RNA, 22 nt, synthetic construct
  source 1..22: mol_type = other RNA
cgaaatggatatttctaattat

SEQ ID NO: 26 - RNA, 22 nt, synthetic construct
  source 1..22: mol_type = other RNA
ataattagaaatatccatttcc

SEQ ID NO: 27 - RNA, 22 nt, synthetic construct
  source 1..22: mol_type = other RNA
aaccctaaatgttatgagagat

SEQ ID NO: 28 - RNA, 22 nt, synthetic construct
  source 1..22: mol_type = other RNA
atctctcataacatttagggtg

SEQ ID NO: 29 - protein, 427 aa, synthetic construct
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 30 - protein, 427 aa, synthetic construct
TQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 31 - protein, 427 aa, synthetic construct
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYSVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 32 - protein, 427 aa, synthetic construct
TQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVNEKYLSEFCFSGTYILSLLLQGYHFTADSWEHIHFIGKIQGSDAGWTLGYMLNLTNMIPAEQPLSTPLSHST

SEQ ID NO: 33 - protein, 427 aa, synthetic construct
TQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEFCFSGTYILSLLLQGYHFTADSWEHIHFIGKIQGSDAGWTLGYMLNLTNMIPAEQPLSTPLSHST

SEQ ID NO: 34 - protein, 427 aa, synthetic construct
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEFCFSGTYILSLLQQGYHFTADSWEHIHFIGKIQGSDAGWTLGYMLNLTNMIPAEQPLSTPLSHST

SEQ ID NO: 35 - protein, 427 aa, synthetic construct
TQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEFCFSGTYILSLLQQGYHFTADSWEHIHFIGKIQGSDAGWTLGYMLNLTNMIPAEQPLSTPLSHST

SEQ ID NO: 36 - protein, 427 aa, synthetic construct
TQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYSVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 37 - protein, 433 aa, synthetic construct
APTSSSTQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEFCFSGTYILSLLLQGYHFTADSWEHIHFIGKIQGSDAGWTLGYMLNLTNMIPAEQPLSTPLSHST

SEQ ID NO: 38 - protein, 433 aa, synthetic construct
APTSSSTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEFCFSGTYILSLLQQGYHFTADSWEHIHFIGKIQGSDAGWTLGYMLNLTNMIPAEQPLSTPLSHST

SEQ ID NO: 39 - protein, 433 aa, synthetic construct
APTSSSTQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEFCFSGTYILSLLQQGYHFTADSWEHIHFIGKIQGSDAGWTLGYMLNLTNMIPAEQPLSTPLSHST

SEQ ID NO: 40 - protein, 430 aa, synthetic construct
APTTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEFCFSGTYILSLLQQGYHFTADSWEHIHFIGKIQGSDAGWTLGYMLNLTNMIPAEQPLSTPLSHST

SEQ ID NO: 41 - protein, 430 aa, synthetic construct
APTTQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEFCFSGTYILSLLQQGYHFTADSWEHIHFIGKIQGSDAGWTLGYMLNLTNMIPAEQPLSTPLSHST

SEQ ID NO: 42 - protein, 430 aa, synthetic construct
APTTQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 43 - protein, 430 aa, synthetic construct
APTTQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYSVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 44 - protein, 443 aa, synthetic construct
APTSSSTKKTQLTSSGTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 45 - protein, 442 aa, synthetic construct
APTSSSTKKTQLTSSTQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVNEKYLSEFCFSGTYILSLLLQGYHFTADSWEHIHFIGKIQGSDAGWTLGYMLNLTNMIPAEQPLSTPLSHST

SEQ ID NO: 46 - protein, 433 aa, synthetic construct
APTSSSTQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYSVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 47 - protein, 430 aa, synthetic construct
APTTQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEFCFSGTYILSLLLQGYHFTADSWEHIHFIGKIQGSDAGWTLGYMLNLTNMIPAEQPLSTPLSHST

SEQ ID NO: 48 - protein, 430 aa, synthetic construct
APTTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 49 - protein, 430 aa, synthetic construct
APTTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYSVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 50 - protein, 510 aa, Homo sapiens
MEDTKESNVKTFCSKNILAILGFSSIIAVIALLAVGLTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYVFLMVLFSLVLFTVAIIGLLIFHKPSYFWKDMV

SEQ ID NO: 51 - RNA, 23 nt, synthetic construct
  source 1..23: mol_type = other RNA
tcttgcggagaactgcatggtgg

SEQ ID NO: 52 - RNA, 23 nt, synthetic construct
  source 1..23: mol_type = other RNA
atctatgtgctggcccagccagg

SEQ ID NO: 53 - RNA, 23 nt, synthetic construct
  source 1..23: mol_type = other RNA
ggagaataaaccagaaagggggg

SEQ ID NO: 54 - RNA, 23 nt, synthetic construct
  source 1..23: mol_type = other RNA
gctactcatttaccgccaagagg

(19) United States
(12) Patent Application Publication
(10) Pub. No.: US 2026/0317737 A1
(43) Pub. Date: Oct. 15, 2026

(54) IMPROVED PRODUCTION OF CD39 VARIANTS

(71) Applicant: NOVARTIS AG, Basel (CH)

(72) Inventors: David AUSLAENDER, Rhein (DE); Nicolas LEBESGUE, Basel, Basel Stadt (CH); Petr OBRDLIK, Freiburg im Breisgau (DE); Joël Aloïs René TAPPAREL, Allschwil, Basel Land (CH); Nina WAGNER, Basel (CH)

(21) Appl. No.: 18/998,832

(22) PCT Filed: Jul. 27, 2023
(86) PCT No.: PCT/IB2023/057614

§ 371 Date: Jan. 27, 2025

Related U.S. Application Data
(60) Provisional application No. 63/369,841, filed on Jul. 29, 2022.
(60) Provisional application No. 63/512,316, filed on Jul. 7, 2023.

(51) Int. Cl. C12N 9/14; A61K 38/00; C12N 5/071; C12N 15/113; C12N 15/85

(52) U.S. Cl. CPC C12N 9/14; C12N 5/0682; C12N 15/1136; C12N 15/85; C12Y 306/01005; A61K 38/00; C12N 2310/141; C12N 2510/02

(57) ABSTRACT

The present invention pertains to the use of miRNA technology for improving recombinant production of CD39 or variants thereof in CHO cells. The miRNA is used for knockdown of the endogenous protein CCL2 of the CHO cells which is difficult to separate from CD39 or variants thereof during purification.

### FIELD OF THE INVENTION

The present invention pertains to the field of recombinant protein production. The present invention provides methods for producing CD39 and variants thereof wherein the level of a host cell protein is reduced which interferes with purification of CD39 and its variants. The production of the interfering host cell protein is reduced using artificial miRNAs targeting the host cell protein.

### BACKGROUND OF THE INVENTION

The generation of recombinant cell lines for production of secreted proteins requires the transfection of a DNA vector into host cells and uses selection markers to enrich stable transfectants. The secreted proteins may affect cell parameters, such as growth, viability and/or productivity, which often requires cell line engineering methods to achieve high-expressing stable cell lines.

Similarly, the quality of the secreted recombinant protein can be affected by intrinsic cell-derived factors. Often, an endogenously expressed protein, such as a cell surface receptor, an enzyme or a protease (host cell proteins, HCP), can be identified as the root cause of the undesired effect impacting the quality of the recombinant protein (see, e.g., WO 2014/097113 A2). In addition, host cell proteins are well known process-related impurities of biologics. When present in drug products, these impurities can cause adverse effects due to increased drug immunogenicity, inflammation and other activities associated with the specific residual host cell protein function (Vanderlaan et al., 2018, Biotechnology Progress 34, 828-837). The composition and abundance of HCPs present in various steps of manufacturing processes and in the final drug substance depend on many factors. These factors can be quite difficult to predict a priori and are often learned only by testing during process development.

Respective undesired effects include enzymatic cleavage of the polypeptide chain of the recombinant protein, such as digestion of the entire protein or amino acid clipping, i.e. removal of one or several amino acids from the N or C terminal end of the protein of interest. Other effects are unwanted post-translational modifications- or removal of desired modifications. Furthermore, endogenous gene products of the host cell may specifically interact with the protein of interest. Such an interaction may recruit the protein of interest from the supernatant of the cell culture, render it difficult to remove the endogenous protein during purification, or even lead to activation of signaling pathways in the host cells which result in reduced growth, viability and/or productivity of the cells. In other cases, the endogenous gene product might simply have chemical and physical properties which are highly similar to the protein of interest which makes it hard to develop a purification process which efficiently removes the host cell protein without diminishing the yield of the protein of interest. This is especially relevant for therapeutic proteins where a high purity and low residual levels of host cell proteins in the final product are a prerequisite for obtaining and maintaining marketing authorization.

Thus, endogenous gene products of the host cell may significantly interfere with the recombinant production of a protein of interest. In view of the above, there is a need in the art to provide strategies to reduce unwanted influence of host cell proteins on the recombinant production of proteins of interest.

### SUMMARY OF THE INVENTION

The present inventors aimed at providing a purification method for human CD39 and variants thereof produced in CHO cells, especially soluble variants of CD39 wherein the membrane anchor sequences are deleted. During development of the purification method, CCL2 was identified as a host cell protein which has physicochemical properties highly similar to those of the CD39 variants and therefore, is not significantly removed during the purification steps. The present inventors therefore established a production cell line for the CD39 variants wherein the CCL2 level is reduced by use of a miRNA against this host cell protein. Thereby, purity of the final CD39 variants could be improved significantly.

In view of this, in a first aspect, the present invention is directed to a method of producing CD39 or a variant thereof, comprising the steps of
(a) providing CHO cells capable of producing CD39 or a variant thereof;
(b) cultivating the CHO cells in a cell culture under conditions which allow for the production of CD39 or the variant thereof;
(c) obtaining CD39 or the variant thereof from the cell culture; and
(d) optionally processing CD39 or the variant thereof;
   wherein the CHO cells are engineered to reduce the production of CCL2 in the CHO cell.

In a second aspect, the present invention provides a CHO cell which is capable of producing CD39 or a variant thereof and which is engineered to reduce the production of CCL2 in the CHO cell.

In certain embodiments of the above aspects, the CHO cells produce a miRNA targeting CCL2. In particular, the CHO cells are engineered by introduction of a vector nucleic acid according to the fifth aspect. In further embodiments of the above aspects, the CHO cells are engineered by knockout of the CCL2 gene.

In a third aspect, the present invention provides a method of improving production of CD39 or a variant thereof in a CHO cell, comprising the steps of
(a-i) providing a CHO cell capable of producing CD39 or a variant thereof;
(a-ii) engineering the CHO cell so as to reduce the production of CCL2 in the CHO cell.

In certain embodiments, step (a-ii) of engineering the CHO cell to reduce the production of CCL2 includes introducing a vector nucleic acid according to the fifth aspect into the CHO cell.

In a fourth aspect, the present invention provides an expression cassette for expression of a miRNA in a CHO cell, comprising a template sequence for a pri-miRNA, wherein the pri-miRNA is suitable to be processed in a CHO cell to form a miRNA targeting CCL2.

In a fifth aspect, the present invention provides a vector nucleic acid for transfection of a CHO cell, comprising the expression cassette according to the fourth aspect.

In a sixth aspect, the present invention provides a method for producing a CHO cell which is capable of expressing CD39 or a variant thereof, comprising introducing a vector nucleic acid according to the fifth aspect into a CHO cell. The vector nucleic acid may additionally comprise a coding sequence encoding CD39 or a variant thereof, or another nucleic acid comprising a coding sequence encoding CD39 or a variant thereof is present in or introduced into the CHO cell.

In a seventh aspect, the present invention provides a composition comprising CD39 or a variant thereof and CCL2, wherein the composition is obtained by production using CHO cells according to the second aspect, and wherein
(i) the amount of CCL2 in the composition is at least 10-times lower compared to the same composition obtained by production using CHO cells which were not engineered to reduce the production of CCL2 in the CHO cells; and/or
(ii) the composition comprises CCL2 in a concentration of 100 ppm or lower, preferably 25 ppm or lower, more preferably 10 ppm or lower.

In an eighth aspect, the present invention provides a CHO cell which is engineered to reduce the production of CCL2 in the CHO cell. In certain embodiments, the CHO cell produces a miRNA targeting CCL2. In particular, the CHO cell is engineered by introduction of a vector nucleic acid according to the fifth aspect. In further embodiments of the above aspects, the CHO cells are engineered by knockout of the CCL2 gene.

Other objects, features, advantages and aspects of the present invention will become apparent to those skilled in the art from the following description and appended claims. It should be understood, however, that the following description, appended claims, and specific examples, which indicate preferred embodiments of the application, are given by way of illustration only. Various changes and modifications within the spirit and scope of the disclosed invention will become readily apparent to those skilled in the art from reading the following.

### Definitions

As used herein, the following expressions are generally intended to preferably have the meanings as set forth below, except to the extent that the context in which they are used indicates otherwise.

The expression “comprise”, as used herein, besides its literal meaning also includes and specifically refers to the expressions “consist essentially of” and “consist of”. Thus, the expression “comprise” refers to embodiments wherein the subject-matter which “comprises” specifically listed elements does not comprise further elements as well as embodiments wherein the subject-matter which “comprises” specifically listed elements may and/or indeed does encompass further elements. Likewise, the expression “have” is to be understood as the expression “comprise”, also including and specifically referring to the expressions “consist essentially of” and “consist of”. The term “consist essentially of”, where possible, in particular refers to embodiments wherein the subject-matter comprises 20% or less, in particular 15% or less, 10% or less or especially 5% or less further elements in addition to the specifically listed elements of which the subject-matter consists essentially of.

The term “nucleic acid” includes single-stranded and double-stranded nucleic acids and ribonucleic acids as well as deoxyribonucleic acids. It may comprise naturally occurring as well as synthetic nucleotides and can be naturally or synthetically modified, for example by methylation, 5′- and/or 3′-capping. In specific embodiments, a nucleic acid refers to a double-stranded deoxyribonucleic acid.

The term “expression cassette” in particular refers to a nucleic acid construct which is capable of enabling and regulating the expression of a coding nucleic acid sequence and/or template nucleic acid sequence introduced therein. An expression cassette may comprise promoters, ribosome binding sites, enhancers and other control elements which regulate transcription of a gene or translation of an mRNA. The exact structure of an expression cassette may vary as a function of the species or cell type, but generally comprises 5′-untranscribed and 5′- and 3′-untranslated sequences which are involved in initiation of transcription and translation, respectively, such as TATA box, capping sequence, CAAT sequence, and the like. More specifically, 5′-untranscribed expression control sequences comprise a promoter region which includes a promoter sequence for transcriptional control of the operatively connected nucleic acid. Expression cassettes may also comprise enhancer sequences or upstream activator sequences. Some expression cassettes are only used for transcription of a template nucleic acid sequence into an RNA product such as a pri-miRNA. Such expression cassettes do not necessarily comprise regulatory elements for translation.

A template nucleic acid is understood according to this application as a DNA which is transcribed into a functional RNA product or a precursor thereof, especially a pri-miRNA. A functional RNA product in particular has a biological activity, alone or in combination with other RNA products and/or proteins, such as the activity of a miRNA (in combination with the proteins of the RISC) to interfere with expression of a target gene.

According to the invention, the term “promoter” refers to a nucleic acid sequence which is located upstream (5′) of the nucleic acid sequence which is to be expressed and controls expression of the sequence by providing a recognition and binding site for RNA-polymerases. The “promoter” may include further recognition and binding sites for further factors which are involved in the regulation of transcription of a gene. A promoter may control the transcription of a prokaryotic or eukaryotic gene. Furthermore, a promoter may be “inducible”, i.e. initiate transcription in response to an inducing agent, or may be “constitutive” if transcription is not controlled by an inducing agent. A gene which is under the control of an inducible promoter is not expressed or only expressed to a small extent if an inducing agent is absent. In the presence of the inducing agent the gene is switched on or the level of transcription is increased. This is mediated, in general, by binding of a specific transcription factor.

The term “vector” is used here in its most general meaning and comprises any intermediary vehicle for a nucleic acid which enables said nucleic acid, for example, to be introduced into prokaryotic and/or eukaryotic cells and, where appropriate, to be integrated into a genome. Vectors of this kind are preferably replicated and/or expressed in the cells. Vectors comprise plasmids, phagemids, bacteriophages or viral genomes. The term “plasmid” as used herein generally relates to a construct of extrachromosomal genetic material, usually a circular DNA duplex, which can replicate independently of chromosomal DNA. The vector according to the present invention may be present in circular or linearized form. A “vector nucleic acid” as used herein is a nucleic acid which forms a vector or is the nucleic acid part of a vector.

The terms “5′” and “3′” is a convention used to describe features of a nucleic acid sequence related to either the position of genetic elements and/or the direction of events (5′ to 3′), such as e.g. transcription by RNA polymerase or translation by the ribosome which proceeds in 5′ to 3′ direction. Synonyms are upstream (5′) and downstream (3′). Conventionally, DNA sequences, gene maps, vector cards and RNA sequences are drawn with 5′ to 3′ from left to right or the 5′ to 3′ direction is indicated with arrows, wherein the arrowhead points in the 3′ direction. Accordingly, 5′ (upstream) indicates genetic elements positioned towards the left hand side, and 3′ (downstream) indicates genetic elements positioned towards the right hand side, when following this convention.

A “polypeptide” or “polypeptide chain” refers to a molecule comprising a polymer of amino acids linked together by peptide bonds. Polypeptides include polypeptides of any length, including proteins (for example, having more than 50 amino acids) and peptides (for example, having 2-49 amino acids). Especially, a polypeptide or polypeptide chain can be a part of a protein which consists of two or more polypeptide chains. Polypeptides include proteins and/or peptides of any activity or bioactivity. The polypeptide can be a pharmaceutically or therapeutically active compound, or a research tool to be utilized in assays and the like.

A target amino acid sequence is “derived” from or “corresponds” to a reference amino acid sequence if the target amino acid sequence shares an identity over its entire length with the reference amino acid sequence of at least 75%, more preferably at least 80%, at least 85%, at least 90%, at least 93%, at least 95%, at least 97%, at least 98% or at least 99%. In particular embodiments, a target amino acid sequence which is “derived” from or “corresponds” to a reference amino acid sequence is 100% identical over its entire length with the reference amino acid sequence. Similarly, a target nucleotide sequence is “derived” from or “corresponds” to a reference nucleotide sequence if the target nucleotide sequence shares an identity over its entire length with the reference nucleotide sequence of at least 75%, more preferably at least 80%, at least 85%, at least 90%, at least 93%, at least 95%, at least 97%, at least 98% or at least 99%. In particular embodiments, a target nucleotide sequence which is “derived” from or “corresponds” to a reference nucleotide sequence is 100% identical over its entire length with the reference nucleotide sequence. An “identity” of an amino acid sequence or nucleotide sequence is preferably determined according to the invention over the entire length of the reference sequence.

As used herein, a “microRNA”, abbreviated “miRNA”, is a single-stranded non-coding RNA molecule which plays a role in RNA silencing and post-transcriptional regulation of gene expression. miRNAs generally consist of 19 to 24 nucleotides, in particular about 22 nucleotides, especially 22 nucleotides. miRNA molecules are capable of silencing mRNAs comprising a complementary nucleotide sequence. Silencing of the target mRNA may occur by cleavage of the mRNA, destabilization of the mRNA or blockage of translation of the mRNA. Silencing of a target mRNA results in reduced or abolished production of the protein encoded by the target mRNA. It is generally understood in the art that the miRNA associates with dicer and argonaute proteins, forming an RNA-induced silencing complex (RISC) which binds to the target mRNA.

miRNAs are produced by transcription of a template DNA sequence into a miRNA precursor (pri-miRNA). The pri-miRNA contains a hairpin stem-loop structure with a double-stranded stem connected to a loop on one side and flanked by single-stranded 5′ and 3′ extensions on the other side. The double-stranded stem contains the guide strand, which forms the miRNA once processed, and the passenger strand which is essentially complementary to the guide strand. Especially, the passenger strand and the guide strand are complementary to each other except for the nucleotide pair at the end of the hairpin stem-loop structure, i.e. the nucleotide pair of passenger and guide strand which is furthest from the loop structure. Guide strand and passenger strand generally each have a length of about 19 to 24 nucleotides, especially of 22 nucleotides. The remaining parts of the pri-miRNA are referred to herein as miRNA scaffold. From 5′ to 3′, the pri-miRNA thus comprises (i) the 5′ miRNA scaffold stem, consisting of the 5′ single-stranded extension and the 5′ part of the stem structure up to the passenger strand; (ii) the passenger strand; (iii) the miRNA scaffold loop; (iv) the guide strand; and (v) the 3′ miRNA scaffold stem, consisting of the 3′ part of the stem structure following the guide strand and the 3′ single-stranded extension. Positions of the passenger strand and the guide strand may also be switched.

The pri-miRNA is processed by cleaving off the 5′ and 3′ miRNA scaffold stems, resulting in a hairpin structure termed pre-miRNA. From 5′ to 3′, the pre-miRNA consists of the passenger strand, the miRNA scaffold loop, and the guide strand; wherein the positions of the passenger strand and the guide strand may also be switched. Then the loop structure is cleaved off and the resulting RNA duplex is separated into the two single-stranded RNA molecules, the guide strand and the passenger strand. The guide strand which is complementary to the targeted mRNA molecule forms the RISC, while the passenger strand generally does not have any function.

The cells referred to herein in particular are host cells. According to the invention, the term “host cell” relates to any cell which can be transformed or transfected with an exogenous nucleic acid. Particular preference is given to mammalian cells such as cells from humans, mice, hamsters, pigs, goats, or primates. The cells may be derived from a multiplicity of tissue types and comprise primary cells and cell lines. A nucleic acid may be present in the host cell in the form of a single copy or of two or more copies and, in one embodiment, is expressed in the host cell. A host cell in particular refers to a cell present in cell culture, especially a cell not present in a living multicellular organism.

The term “CD39” as used herein refers to the human protein CD39 (Cluster of Differentiation 39), also known as ectonucleoside triphosphate diphosphohydrolase-1 (NTPDase1 or Ecto-ATPDase 1). CD39 is an ectonucleotidase present on the cell surface with a catalytic site on the extracellular face that catalyzes the hydrolysis of γ- and β-phosphate residues of triphospho- and diphosphonucleosides to the monophosphonucleoside derivative. Human CD39 in particular has the amino acid sequence of SEQ ID NO: 50 and/or is represented by UniProt entry P49961.

A “variant of CD39” refers to a protein derived from human CD39 which has an amino acid sequence being at least 60% identical to human CD39 over the entire length of human CD39. In certain embodiments, the variant of CD39 only consists of the extracellular domain or a part thereof of human CD39 (amino acids 38 to 478 of SEQ ID NO: 50). In particular, the variant of CD39 has an amino acid sequence being at least 80% identical to the extracellular domain of human CD39 over the entire length of amino acid positions 38 to 478 of human CD39. In particular, the variant of CD39 is a soluble variant, i.e. a variant which lacks the transmembrane domains of human CD39 and its membrane anchors. In certain embodiments, the variant has an N terminal deletion of 30 to 50 amino acids, a C terminal deletion of 20 to 40 amino acids and/or a central deletion of 10 to 15 amino acids compared to human CD39. In addition, the variant may comprise up to 5 point mutations compared to human CD39. Certain variants of CD39 are disclosed in WO 2020/016804. In particular, the variant of CD39 has an amino acid sequence selected from the group consisting of SEQ ID NOs: 29 to 49.

The term “CCL2” as used herein refers to the Chinese hamster protein CCL2 (C—C motif chemokine receptor ligand 2), also known as monocyte chemoattractant protein 1 (MCP1). CCL2 in particular has the amino acid sequence of SEQ ID NO: 24. Amounts and concentrations of CCL2 in a composition as referred to herein are in particular determined by ELISA (enzyme-linked immune adsorbent assay) or LC-MS (liquid chromatography-mass spectrometry, especially by LC-MS. Respective measurements are preferably performed as described in example 5, sections 5 and 6, respectively.

The term “pharmaceutical composition” or “pharmaceutical formulation” particularly refers to a composition suitable for administering to a human or animal, i.e., a composition containing components which are pharmaceutically acceptable. Preferably, a pharmaceutical composition comprises an active compound or a salt or prodrug thereof together with a carrier, diluent or pharmaceutical excipient such as buffer, preservative and tonicity modifier.

The numbers given herein may in certain embodiments be understood as approximate numbers. In particular, the numbers preferably may be up to 10% higher and/or lower, in particular up to 9%, 8%, 7%, 6%, 5%, 4%, 3%, 2% or 1% higher and/or lower. In specific embodiments, the umbers given herein are not approximate numbers and may only vary within the inaccuracy of the technical measurement.

Numeric ranges described herein are inclusive of the numbers defining the range. The headings provided herein are not limitations of the various aspects or embodiments of this invention which can be read by reference to the specification as a whole. According to one embodiment, subject-matter described herein as comprising certain steps in the case of methods or as comprising certain ingredients in the case of compositions refers to subject-matter consisting of the respective steps or ingredients. It is preferred to select and combine preferred aspects and embodiments described herein and the specific subject-matter arising from a respective combination of preferred embodiments also belongs to the present disclosure.

### DETAILED DESCRIPTION OF THE INVENTION

The present invention is based on the finding that the endogenous CHO protein CCL2 causes major problems during purification of recombinantly produced therapeutic CD39 variants. Typically, residual host proteins are removed from the drug by adapting or by optimizing downstream purification process, e.g. by introducing novel or optimized affinity purification steps. As observed during the purification process, the host cell protein CCL2 is not separated from the CD39 variants by the established chromatography steps, especially by anion exchange chromatography or hydrophobic interaction chromatography. It is assumed that this lack of separation is caused by highly similar physicochemical properties of CCL2 and the CD39 variants, in particular by a similar charge distribution.

The present inventors solved this problem by knocking down CCL2 in the CHO host cells using a miRNA approach. Such approach has the advantage that the presence of the problematic protein can avoid from the very beginning of the production process and does not require further timely and potentially costly adaptions of downstream process. Transfecting the CHO host cell with a vector producing a miRNA targeting the CCL2 mRNA showed a strong knock-down efficacy and succeeded in significantly reducing the CCL2 level in the cell culture supernatant. Thereby, in the final composition of the purified CD39 variants CCL2 was lowered to an insignificant residual amount. Furthermore, the method of producing CD39 or a variant thereof as described herein, allow to produce a pure, and safe CD39 or a variant thereof, suitable for use as a drug.

### 1. Production Methods for CD39 and Variants Thereof

In a first aspect, the present invention provides a method of producing CD39 or a variant thereof, comprising the steps of
(a) providing CHO cells capable of producing CD39 or a variant thereof;
(b) cultivating the CHO cells in a cell culture under conditions which allow for the production of CD39 or the variant thereof;
(c) obtaining CD39 or the variant thereof from the cell culture; and
(d) optionally processing CD39 or the variant thereof;
   wherein the CHO cells are engineered to reduce the production of CCL2 in the CHO cell.

The steps of the method are generally performed in the indicated order.

In specific embodiments, the CHO cells are engineered to reduce the production of CCL2 in the CHO cell. The CHO cells may be engineered in any suitable way to reduce the production of CCL2. Reducing the production of CCL2 in particular refers to reducing the expression of CCL2. In general, cell line engineering, especially genetic engineering, is used. In certain embodiments, production of CCL2 in the CHO cells is reduced by knockdown or knockout of CCL2 expression or degradation of CCL2 protein.

Knockdown of CCL2 expression can be done by reducing the amount of functional mRNA encoding CCL2 in the cell and/or reducing the rate of translation of the mRNA encoding CCL2 into CCL2 protein. In specific embodiments, the CHO cells are engineered to produce a miRNA targeting the endogenous CCL2 of the CHO cells.

Knockout of CCL2 expression may be performed by deleting one or both alleles of the gene encoding CCL2 in the genome of the CHO cell or mutating one or both of these alleles so that they cannot be transcribed into functional mRNA. Knockout of CCL2 expression may be done using, for example, homologous recombination, site-specific nucleases, CRISPR/Cas, TALENs or Cas-Clover.

Reducing of the production of CCL2 in particular means that after engineering of the CHO cell, the amount of CCL2 in the cell is 50% or less of the amount of CCL2 in the cell prior to engineering. In certain embodiments, the amount of CCL2 in the cell is 25% or less, especially 10% or less or 5% or less of the amount of CCL2 in the cell prior to engineering.

In a further aspect, the present invention provides a method of producing CD39 or a variant thereof, comprising the steps of
(a) providing host cells capable of producing CD39 or a variant thereof;
(b) cultivating the host cells in a cell culture under conditions which allow for the production of CD39 or the variant thereof;
(c) obtaining CD39 or the variant thereof from the cell culture; and
(d) optionally processing CD39 or the variant thereof;
   wherein the host cells are engineered to reduce the production of CCL2 in the host cell.

The host cell may be any cell suitable for producing CD39 or a variant thereof, especially human CD39 or a variant thereof, and endogenously expressing CCL2 when not engineered to reduce the production of CCL2. Especially, the host cell is a mammalian cell. It may be selected from, but not limited to, the group consisting of cells derived from mice, such as COP, L, C127, Sp2/0, NS0, NS1, At20 and NIH3T3; rats, such as PC12, PC12h, GH3, MIT, YB2/0 and Y0; hamsters, such as BHK, CHO and DHFR gene defective CHO; monkeys, such as COS1, COS3, COS7, CV1 and Vero; and humans, such as Hela, HEK293, CAP, retina-derived PER-C6, cells derived from diploid fibroblasts, myeloma cells and HepG2. In specific embodiments, the host cell is a Chinese hamster ovary (CHO) cell. The host cell may be suitable for suspension cultures and/or adherent cultures, and in particular can be used in suspension cultures. The features, embodiments and examples of the method of producing CD39 or a variant thereof according to the first aspect of the invention also likewise apply to the method according to this further aspect of the invention.

1.1 the miRNA Targeting CCL2

In certain embodiments, the CHO cells are engineered to produce a miRNA targeting the endogenous CCL2 of the CHO cells. It is understood that the miRNA targeting CCL2 is at least partially complementary to and is capable of binding to and initiating silencing of the mRNA or pre-mRNA encoding CCL2. Silencing may occur via degradation of the targeted mRNA or preventing the targeted mRNA from being translated. The miRNA may be complementary to a part of the 5′ UTR, to a part of the coding region, to a part of an intron, and/or to a part of the 3′ UTR of the CCL2 mRNA. In particular, the miRNA targeting CCL2 is complementary to a part of the 3′UTR of the CCL2 mRNA. Exemplary miRNAs targeting CCL2 have a nucleotide sequence selected from the group consisting of SEQ ID NOs: 26 and 28.

In specific embodiments, the CHO cells comprise an expression cassette for production of the miRNA targeting CCL2. The expression cassette may be present on a plasmid in the cell or may be integrated into the genome of the cell. The expression cassette comprises a template sequence for a pri-miRNA, wherein the pri-miRNA is suitable to be processed in the CHO cell to form the miRNA targeting CCL2. The expression cassette enables transcription of the template sequence into the pri-RNA. the template sequence may be present anywhere within the transcribed region of the expression cassette.

In certain embodiments, the template sequence is present within an intronic sequence. Upon transcription, the intronic sequence is excised from the transcribed RNA, thereby forming the pri-miRNA.

Since the expression cassette comprises the template sequence for the pri-miRNA within an intronic sequence, it may contain further sequences for expression of other products, such as coding sequences for the production of a polypeptide of interest, coding sequences for the production of selectable marker, and template sequence for other RNA products, especially other pri-miRNAs. Alternatively, the expression cassette may be used exclusively for production of the miRNA targeting CCL2.

The expression cassette may comprise the template sequence for the pri-miRNA within an intronic sequence. Upon expression, a pre-mRNA is formed which contains the intronic sequence. The intronic sequence is then spliced out of the pre-mRNA, thereby forming the pri-miRNA which thereafter is further processed to ultimately provide the miRNA. The formed pre-mRNA does not have to comprise any sequences coding for a polypeptide.

In certain embodiments, the expression cassette further comprises a polymerase II promoter. This promoter is functionally linked to the template sequence for the pri-miRNA and controls expression of the pri-miRNA. The promoter may be any RNA polymerase II promoter suitable for expression of a gene in a host cell, especially a CHO cell. For example, the promoter may be selected from the group consisting of cytomegalovirus (CMV) promoter, simian virus 40 (SV40) promoter, ubiquitin C (UBC) promoter, elongation factor 1 alpha (EF1A) promoter, phosphoglycerate kinase (PGK) promoter, Rous sarcoma virus (RSV) promoter, BROAD3 promoter, murine rosa 26 promoter, pCEFL promoter, chicken β-actin promoter (CBA), B-actin promoter coupled with CMV early enhancer (CAGG), a-1-antitrypsin promoter, and inducible promoters such as tetracycline-inducible promoters (e.g. pTRE), and vanillic acid inducible promoters. In specific embodiments, the promoter is a CMV promoter or a SV40 promoter, especially a CMV promoter.

In certain embodiments, the expression cassette further comprises a terminator. The terminator is functionally linked to the template sequence for the pri-miRNA and controls expression of the pri-miRNA. The term “terminator” as used herein refers to a transcription terminator which terminates transcription of the DNA into RNA, especially by RNA polymerase II.

The template sequence for the pri-miRNA is in particular located between the promoter and the terminator of the expression cassette.

In certain embodiments, the expression cassette comprises a coding sequence encoding, for example, a polypeptide of interest or a selectable marker. In these embodiments, the expression cassette may further comprise a 5′ untranslated region (5′UTR) and a 3′ untranslated region (3′UTR). The intronic sequence comprising the template sequence for the pri-miRNA may be present within the 5′UTR, the 3′UTR or the coding sequence. In particular, the intronic sequence is present within the 5′UTR or the 3′UTR, especially within the 5′UTR. In alternative embodiments, the expression cassette does not comprise a coding sequence encoding a polypeptide.

The intronic sequence comprising the template sequence for the pri-miRNA in particular comprises a splice donor site upstream of the pri-miRNA and a corresponding splice acceptor site downstream of the pri-miRNA. With these splice donor and acceptor sites, the pri-miRNA is spliced out of the pre-mRNA after transcription.

In certain embodiments, the intronic sequence comprising two or more template sequences for a pri-miRNA. In these embodiments, the intronic sequence comprises a splice donor site upstream of the template sequence for the first pri-miRNA, i.e. the most 5′ template sequence, and a corresponding splice acceptor site downstream of the template sequence for the last pri-miRNA, i.e. the most 3′ template sequence. Adjacent template sequences within an intronic sequence may be separated from each other by a spacer sequence. Such a spacer sequence in particular forms a RNA stem loop structure, such as the sequence of SEQ ID NO: 22.

In certain embodiments, the expression cassette comprises only one intronic sequence with one or more template sequences for a pri-miRNA. In alternative embodiments, the expression cassette comprises two or more intronic sequence with one or more template sequences for a pri-miRNA.

The pri-miRNAs of the two or more template sequences present within the same or different intronic sequences in particular are different from each other. In specific embodiments, the miRNAs produced from the pri-miRNAs all target CCL2, but bind to different parts of the mRNA or pre-mRNA of CCL2.

The expression cassette may comprise a coding sequence which encodes a polypeptide. The coding sequence may in particular code for a selectable marker. The coding sequence preferably is functionally linked to the polymerase II promoter and the terminator of the expression cassette. In embodiments wherein the expression cassette comprises the template sequence for the pri-miRNA and the coding sequence for the selectable marker, the expression of the miRNA is linked to the selectable marker expression. Thus, by increasing the selection pressure during clone selection, also the miRNA level is increased.

The selectable marker may be selected from the group consisting of folate receptor (FAR), dihydrofolate reductase (DHFR), glutamine synthetase, puromycin, hygromycin, neomycin, zeocin, and blasticidin. In certain embodiments, the selectable marker is a folate receptor (FAR).

The expression cassette comprises a template sequence for the pri-miRNA. The pri-miRNA produced from the expression cassette may have any structure suitable for processing by the host cell in order to obtain a functional miRNA which targets CCL2. The functional miRNA induces reduction of the level of CCL2 in the host cell.

In certain embodiments, the pri-miRNA comprises a passenger strand and a guide strand. The guide strand in particular comprises or consists of the miRNA formed after processing of the pri-miRNA by the host cell. The pri-miRNA furthermore, may comprise a miRNA scaffold loop and/or a miRNA scaffold stem, especially a 5′ miRNA scaffold stem and a 3′ miRNA scaffold stem. In specific embodiments, the pri-miRNA comprises, from 5′ to 3′, a 5′ miRNA scaffold stem, a passenger strand, a miRNA scaffold loop, a guide strand, and a 3′ miRNA scaffold stem. In alternative embodiments, the pri-miRNA comprises, from 5′ to 3′, a 5′ miRNA scaffold stem, a guide strand, a miRNA scaffold loop, a passenger strand, and a 3′ miRNA scaffold stem. Embodiments wherein the passenger strand is positioned upstream of the guide strand are preferred.

The passenger strand and the guide strand of the pri-miRNA in particular have artificial sequences. An artificial sequence in this respect refers to a sequence which is not present as passenger or guide strand in naturally occurring miRNAs. In particular, the sequences of the passenger strand and the guide strand are not found in naturally occurring miRNAs.

In certain embodiments, the guide strand has the nucleotide sequence of SEQ ID NO: 26 and the passenger strand has the nucleotide sequence of SEQ ID NO: 25. In other embodiments, the guide strand has the nucleotide sequence of SEQ ID NO: 28 and the passenger strand has the nucleotide sequence of SEQ ID NO: 27.

In certain embodiments, one or more of the scaffold sequences of the pri-miRNA or pre-miRNA are derived from a naturally occurring pri-miRNA, especially a pri-miRNA naturally occurring in mammals, in particular in humans. In specific embodiments, all of the scaffold sequences of the pri-miRNA are derived from a naturally occurring pri-miRNA, especially a pri-miRNA naturally occurring in mammals, in particular in humans. In particular, all of the scaffold sequences of the pri-miRNA are derived from the same naturally occurring pri-miRNA. The scaffold sequences of the pri-miRNA in particular comprise the 5′ miRNA scaffold stem, the miRNA scaffold loop and the 3′ miRNA scaffold stem. Suitable naturally occurring pri-miRNAs from which the scaffold sequences may be derived include miR-30A, miR-E, SIBR, eSIBR, miR-1, miR-155, miR-16, miR-16-1, miR-16-2, miR-3G, miRGE, miR100, miR125b, miR-130a, miR-190a, miR-193a, miR-211, miR-26a, miR-340, miR-7-2, miR-96, and miR-44. Thus, in one embodiments the 5′ miRNA scaffold stem, the miRNA scaffold loop, and the 3′ miRNA scaffold stem are derived from one or more pre-miRNAs selected from the group consisting of miR-30A, miR-E, SIBR, eSIBR, miR-1, miR-155, miR-16, miR-16-1, miR-16-2, miR-3G, miRGE, miR 100, miR125b, miR-130a, miR-190a, miR-193a, miR-211, miR-26a, miR-340, miR-7-2, miR-96, and miR-44. In specific embodiments, the naturally occurring pri-miRNA from which the scaffold sequences are derived is miR-30A.

In specific embodiments, all of the scaffold sequences of the pri-miRNA share a nucleotide sequence identity with the corresponding scaffold sequences of a naturally occurring pri-miRNA of at least 80%, especially at least 90%, in particular at least 95% over their entire length. In certain embodiments, the 5′ miRNA scaffold stem of the pri-miRNA shares a nucleotide sequence identity with the corresponding scaffold sequence of a naturally occurring pri-miRNA of at least 80%, especially at least 85%, in particular at least 90% over its entire length. In certain embodiments, the 3′ miRNA scaffold stem of the pri-miRNA shares a nucleotide sequence identity with the corresponding scaffold sequence of a naturally occurring pri-miRNA of at least 80%, especially at least 90%, in particular at least 95% over its entire length. In certain embodiments, the miRNA scaffold loop of the pri-miRNA shares a nucleotide sequence identity with the corresponding scaffold sequence of a naturally occurring pri-miRNA of at least 60%, especially at least 70%, in particular at least 75% over its entire length. In these embodiments, the naturally occurring pri-miRNA may in particular be miR-30A.

In certain embodiments, the 5′ miRNA scaffold stem of the pri-miRNA comprises the nucleotide sequence of any one of SEQ ID NOs: 1-7 or a sequence derived therefrom. In particular, the 5′ miRNA scaffold stem of the pri-miRNA comprises the nucleotide sequence of any one of SEQ ID NOs: 1-7 or a sequence sharing a nucleotide sequence identity therewith of at least 90%, preferably at least 95%, more preferably at least 98%, and most preferably 100%. Especially, the 5′ miRNA scaffold stem of the pri-miRNA consists of the nucleotide sequence of any one of SEQ ID NOs: 1-4, in particular SEQ ID NO: 1.

In certain embodiments, the miRNA scaffold loop of the pri-miRNA comprises the nucleotide sequence of any one of SEQ ID NOs: 8-10 or a sequence derived therefrom. In particular, the miRNA scaffold loop of the pri-miRNA comprises the nucleotide sequence of any one of SEQ ID NOs: 8-10 or a sequence sharing a nucleotide sequence identity therewith of at least 75%, preferably at least 85%, more preferably at least 90%, and most preferably 100%. Especially, the miRNA scaffold loop of the pri-miRNA consists of the nucleotide sequence of any one of SEQ ID NOs: 8-10, in particular SEQ ID NO: 8.

In certain embodiments, the 3′ miRNA scaffold stem of the pri-miRNA comprises the nucleotide sequence of any one of SEQ ID NOs: 11-17 or a sequence derived therefrom. In particular, the 3′ miRNA scaffold stem of the pri-miRNA comprises the nucleotide sequence of any one of SEQ ID NOs: 11-17 or a sequence sharing a nucleotide sequence identity therewith of at least 90%, preferably at least 95%, more preferably at least 98%, and most preferably 100%. Especially, the 3′ miRNA scaffold stem of the pri-miRNA consists of the nucleotide sequence of any one of SEQ ID NOs: 11-14, in particular SEQ ID NO: 14.

In certain embodiments, the template sequence for the pri-miRNA comprises at least one recognition site, especially two recognition sites, for a DNA restriction enzyme. In particular, the two recognition sites are for different DNA restriction enzymes and generate different overhangs after cleavage. The two recognition sites preferably flank the pre-miRNA part of the pri-miRNA-which comprises the guide strand, the passenger strand and the miRNA scaffold loop-on both sides. In particular, one of the recognition sites is located within the sequence which is transcribed into the 5′ miRNA scaffold stem and the other recognition site is located within the sequence which is transcribed into the 3′ miRNA scaffold stem. In specific embodiments, the recognition sites are located within the sequences which are transcribed into the single-stranded parts of the 5′ and 3′ miRNA scaffold stems. The recognition sites in particular are unique recognition sites within the expression cassette, especially within the entire vector harboring the expression cassette.

### 1.2 Knockout of the CCL2 Gene

In certain embodiments, the CHO cells are engineered by knockout of the CCL2 gene. Knockout of the CCL2 gene refers to deletion or inactivation of the CCL2 gene. In specific embodiments, both alleles of the CCL2 gene in the CHO cells are knocked out. The CCL2 gene may be completely removed, or its transcription may be impaired or the translation of a complete CCL2 protein may be inhibited. In particular, a mutation may be introduced into the gene which results in a frameshift of the coding sequence of the CCL2 mRNA.

Knockout of CCL2 expression may be done using, for example, homologous recombination, site-specific nucleases, CRISPR/Cas, TALENs or Cas-Clover. In particular, knockout using Cas-Clover may be performed using the gRNA pair with a gRNA comprising the nucleotide sequence of SEQ ID NO: 53 and another gRNA comprising the nucleotide sequence of SEQ ID NO: 54.

### 1.3 the Production and Purification Steps

In certain embodiments, the method further comprises between steps (a) and (b) the steps of
(a1) inoculating a cell culture medium with the CHO cells to provide a cell culture, and
(a2) cultivating the CHO cells in the cell culture under conditions which allow for increasing the number of CHO cells in the cell culture.

Suitable conditions for cultivating the host cells, increasing their cell number and expressing CD39 or the variant thereof can be readily determined by the skilled person and are also already known in the art. In certain embodiments, a vector nucleic acid encoding CD39 or the variant thereof in the CHO cells comprises one or more selectable marker genes. In these embodiments, the culturing conditions in step (a2) and/or (b) may include the presence of corresponding selection agent(s) in the cell culture medium.

Obtaining CD39 or the variant thereof from the cell culture in step (c) in particular includes isolating CD39 or the variant thereof from the cell culture. Isolation of CD39 or the variant thereof in particular refers to the separation of CD39 or the variant thereof from the remaining components of the cell culture. The term “cell culture” as used herein in particular includes the cell culture medium and the CHO cells. In certain embodiments, CD39 or the variant thereof is secreted by the CHO cells. In these embodiments, CD39 or the variant thereof is isolated from the cell culture medium. Separation of CD39 or the variant thereof from the cell culture medium may be performed, for example, by chromatographic methods and/or filtration methods.

Suitable methods and means for isolating CD39 or the variant thereof are known in the art and can be readily applied by the skilled person. Exemplary isolation methods for example include filtration steps such as tangential flow filtration, alternating flow filtration, depth filtration, ultrafiltratrion and diafiltration, and/or chromatography steps such as affinity chromatography, anion- and/or cation exchange chromatography, hydrophilic interaction chromatography, size exclusion chromatography and reverse phase chromatography. The step of obtaining CD39 or the variant thereof from the cell culture may in particular include a filtration step, a capture chromatography step and one or more polishing chromatography steps. In addition, step (c) may further include one or more virus inactivation steps.

In specific embodiments, step (c) comprises performing anion exchange chromatography and/or hydrophobic interaction chromatography. In particular, step (c) comprises:
(c1) separating the CHO cells from cell culture supernatant containing CD39 or the variant thereof;
(c2) separating CD39 or the variant thereof from the cell culture supernatant using anion exchange chromatography;
(c3) further purifying CD39 or the variant thereof using hydrophobic interaction chromatography; and (c4) further purifying CD39 or the variant thereof using anion exchange chromatography.

Step (c1) may in particular be performed using a filtration method such as tangential flow filtration or alternating flow filtration, wherein the CHO cells are held back in the retentate and the cell culture supernatant containing CD39 or the variant thereof pass through the filter to the permeate. Furthermore, step (c1) may also be performed using a centrifugation method. In addition to steps (c1) to (c4), step (c) may also further comprise one or more ultrafiltration and/or diafiltration steps for buffer exchange between or after the chromatography steps as well as virus inactivation and/or virus retention steps. Virus inactivation may in particular be achieved using incubation at low pH for a time sufficient to inactivate substantially all viruses, and virus retention may be achieved using sterile filtration.

The obtained CD39 or the variant thereof may optionally be subject to further processing steps (d) such as e.g. modification and/or formulation steps in order to produce CD39 or the variant thereof in the desired quality and composition. Such further processing steps and methods are generally known in the art. Formulation steps may include buffer exchange, addition of formulation components, pH adjustment, and concentration adjustment. Any combination of these and further steps may be used.

In certain embodiments, the method for producing CD39 or a variant thereof further comprises as step (d) or part of step (d) the step of providing a pharmaceutical formulation comprising CD39 or the variant thereof. Providing a pharmaceutical formulation comprising CD39 or the variant thereof or formulating CD39 or the variant thereof as a pharmaceutical composition in particular comprises exchanging the buffer solution or buffer solution components of the composition comprising CD39 or the variant thereof. Furthermore, this step may include lyophilization of CD39 or the variant thereof. In particular, CD39 or the variant thereof is transferred into a composition only comprising pharmaceutically acceptable ingredients.

The method of producing CD39 or a variant thereof in particular provides the CD39 or the variant thereof with a higher purity compared to the same method using CHO cells which are not engineered to reduce the production of CCL2 in the CHO cells. In certain embodiments, CD39 or the variant thereof obtained after step (c) is in a composition which comprises CCL2 in a concentration which is at least 10-times lower, preferably 25-times lower, more preferably 100-times lower, compared to a composition obtained with the same method after step (c) using CHO cells which are not engineered to reduce the production of CCL2 in the CHO cells. In particular, CD39 or the variant thereof obtained after step (c) is in a composition which comprises CCL2 in a concentration of 100 ppm or lower, preferably 25 ppm or lower, more preferably 10 ppm or lower.

In certain embodiments, CD39 or the variant thereof obtained after step (c1) is in a composition which comprises CCL2 in a concentration which is at least 10-times lower, preferably 25-times lower, more preferably 100-times lower, compared to a composition obtained with the same method after step (c1) using CHO cells which are not engineered to reduce the production of CCL2 in the CHO cells. In particular, CD39 or the variant thereof obtained after step (c1) is in a composition which comprises CCL2 in a concentration of 100 ppm or lower, preferably 25 ppm or lower, more preferably 10 ppm or lower.

In certain embodiments, CD39 or the variant thereof obtained after step (c4) is in a composition which comprises CCL2 in a concentration which is at least 10-times lower, preferably 25-times lower, more preferably 100-times lower, compared to a composition obtained with the same method after step (c4) using CHO cells which are not engineered to reduce the production of CCL2 in the CHO cells. In particular, CD39 or the variant thereof obtained after step (c4) is in a composition which comprises CCL2 in a concentration of 25 ppm or lower, preferably 10 ppm or lower, more preferably 5 ppm or lower.

In certain embodiments, CD39 or the variant thereof obtained after step (d) is in a composition which comprises CCL2 in a concentration which is at least 10-times lower, preferably 25-times lower, more preferably 100-times lower, compared to a composition obtained with the same method after step (d) using CHO cells which are not engineered to reduce the production of CCL2 in the CHO cells. In particular, CD39 or the variant thereof obtained after step (d) is in a composition which comprises CCL2 in a concentration of 25 ppm or lower, preferably 10 ppm or lower, more preferably 5 ppm or lower.

### 1.4 CD39 and the Variants Thereof

The CD39 or variant thereof produced by the method in particular is human CD39 or a variant of human CD39. In certain embodiments, the CD39 or the variant thereof does not comprise any membrane anchors. In this embodiment, it is in particular a soluble variant of CD39. In specific embodiments, the variant has N terminal, C terminal and/or central deletions compared to human CD39, especially N terminal, C terminal and central deletions. The N terminal deletion may in particular comprise 30 to 50 amino acids. The C terminal deletion may in particular comprise 20 to 40 amino acids. The central deletion may in particular comprise 10 to 15 amino acids. In addition, the variant in certain embodiments comprises up to 5 point mutations compared to human CD39.

In certain embodiments, the CD39 variant has an amino acid sequence selected from the group consisting of SEQ ID NOs: 29-49. In particular, the CD39 variant comprises the amino acid sequence of SEQ ID NO: 49, and especially consists of the amino acid sequence of SEQ ID NO: 49.

### 2. CHO Host Cells

In a second aspect, the present invention provides a CHO cell which is capable of producing CD39 or a variant thereof and which is engineered to reduce the production of CCL2 in the CHO cell. The CHO cell in particular may be engineered as described herein above with respect to the method of producing CD39 or a variant thereof.

In a further aspect, the present invention provides a host cell which is capable of producing CD39 or a variant thereof and which is engineered to reduce the production of CCL2 in the host cell. The host cell in particular is a host cell as described herein above with respect to the method of producing CD39 or a variant thereof and may be engineered as described herein above with respect to the method of producing CD39 or a variant thereof. The features and embodiments described herein for the CHO cell likewise also apply to the host cell in general.

The host cell may be any cell suitable for producing CD39 or a variant thereof, especially human CD39 or a variant thereof, and endogenously expressing CCL2 when not engineered to reduce the production of CCL2. Especially, the host cell is a mammalian cell. It may be selected from, but not limited to, the group consisting of cells derived from mice, such as COP, L, C127, Sp2/0, NS0, NS1, At20 and NIH3T3; rats, such as PC12, PC12h, GH3, MIT, YB2/0 and Y0; hamsters, such as BHK, CHO and DHFR gene defective CHO; monkeys, such as COS1, COS3, COS7, CV1 and Vero; and humans, such as Hela, HEK293, CAP, retina-derived PER-C6, cells derived from diploid fibroblasts, myeloma cells and HepG2. In specific embodiments, the host cell is a Chinese hamster ovary (CHO) cell. The host cell may be suitable for suspension cultures and/or adherent cultures, and in particular can be used in suspension cultures.

The CHO cell or host cell may contain further exogenous nucleic acids in addition to the expression cassette according to the fourth aspect or the vector nucleic acid according to the fifth aspect of the invention. In particular, the CHO cell or host cell may contain an expression cassette for expression of CD39 or a variant thereof which is not the expression cassette according to the fourth aspect and which is not present on the vector nucleic acid according to the fifth aspect. Said expression cassette for expression of CD39 or a variant thereof may be present on a further vector nucleic acid or integrated into the genome of the CHO cell or host cell.

In certain embodiments, the coding sequence of CD39 or a variant thereof is present in the CHO cell or host cell:
(i) within the expression cassette which expresses the miRNA targeting CCL2,
(ii) within a further expression cassette on the same vector nucleic acid as the expression cassette which expresses the miRNA targeting CCL2, or
(iii) on a further vector nucleic acid or in the genome of the CHO cell or host cell.

The CD39 or the variant thereof in particular is a CD39 or a variant thereof as described herein.

In a further aspect, the present invention provides a mammalian cell, especially a CHO cell, which is engineered to reduce the production of CCL2 in the mammalian cell. The mammalian cell may be engineered as described herein above with respect to the method of producing CD39 or a variant thereof.

In an eighth aspect, the present invention provides a CHO cell which is engineered to reduce the production of CCL2 in the CHO cell. In certain embodiments, the CHO cell produces a miRNA targeting CCL2. In particular, the CHO cell is engineered by introduction of a vector nucleic acid according to the fifth aspect. In further embodiments of the above aspects, the CHO cells are engineered by knockout of the CCL2 gene.

The embodiments, features and examples of the CHO cell according to the second aspect also apply likewise to the CHO cell according to the eighth aspect, except that the CHO cell according to the eighth aspect does not necessarily have to be capable of producing CD39 or a variant thereof.

### 3. Methods for Improving Production of CD39 and Variants Thereof

In a third aspect, the present invention provides a method of improving production of CD39 or a variant thereof in a CHO cell, comprising the steps of
(a-i) providing a CHO cell capable of producing CD39 or a variant thereof;
(a-ii) engineering the CHO cell so as to reduce the production of CCL2 in the CHO cell.

Engineering of the CHO cell may in particular be performed as described herein above with respect to the method of producing CD39 or a variant thereof. In certain embodiments, the CHO cell is engineered in step (a-ii) by knockdown or knockout of CCL2 expression or inducing degradation of CCL2 protein in the CHO cell. Especially, the CHO cell is engineered in step (a-ii) to produce a miRNA targeting the endogenous CCL2 of the CHO cells. In specific embodiments, step (a-ii) of engineering the CHO cell to reduce the production of CCL2 includes introducing a vector nucleic acid encoding a miRNA targeting CCL2 into the CHO cell, especially a vector nucleic acid which comprises an expression cassette for expression of the miRNA targeting CCL2. In particular, a vector nucleic acid as described herein is used.

The embodiments, features and examples of the method of producing CD39 or a variant thereof as described herein also likewise apply to the method of improving production of CD39 or a variant thereof in a CHO cell. In particular, the CHO cell, the CD39 or the variant thereof, the miRNA targeting CCL2 and/or the expression cassette may be as defined herein above with respect to the method of producing CD39 or a variant thereof.

In certain embodiments, step (a-ii) of engineering the CHO cell results in an engineered CHO cell wherein the level of CCL2 mRNA is reduced by at least 5-fold, preferably at least 10-fold, more preferably at least 25-fold, compared to the same CHO cell prior to step (a-ii). In specific embodiments, step (a-ii) of engineering the CHO cell results in an engineered CHO cell wherein the level of CCL2 protein is reduced by at least 5-fold, preferably at least 10-fold, more preferably at least 25-fold, compared to the same CHO cell prior to step (a-ii).

The method of improving production of CD39 or a variant thereof in a CHO cell may further comprise the steps of:
(b) cultivating the CHO cell obtained in step (a-ii) in a cell culture under conditions which allow for proliferation of the CHO cell and simultaneous and/or subsequent production of CD39 or the variant thereof;
(c) obtaining said CD39 or the variant thereof from the cell culture; and
(d) optionally processing CD39 or the variant thereof.

The embodiments, features and examples of steps (a), (b), (c) and (d) of the method of producing CD39 or a variant thereof as described herein also likewise apply to steps (a-i), (b), (c) and (d), respectively, of the method of improving production of CD39 or a variant thereof in a CHO cell.

### 4. Expression Cassettes and Vector Nucleic Acids

In a fourth aspect, the present invention provides an expression cassette for expression of a miRNA in a CHO cell, comprising a template sequence for a pri-miRNA, wherein the pri-miRNA is suitable to be processed in a CHO cell to form a miRNA targeting CCL2.

The features, embodiments and examples of the expression cassette described herein above with respect to the method of producing CD39 or a variant thereof also likewise apply to the expression cassette according to the fourth aspect of the invention.

In a fifth aspect, the present invention provides a vector nucleic acid for transfection of a CHO cell, comprising the expression cassette according to the fourth aspect. The vector nucleic acid may be any vector nucleic acid suitable for transfection of a CHO cell. In certain embodiments, the vector nucleic acid is a plasmid. In other embodiments, the vector nucleic acid is a viral vector.

The vector nucleic acid may comprise further elements in addition to the expression cassette. For example, the vector nucleic acid may comprise an origin of replication (ORI), a coding sequence encoding a polypeptide of interest, a selectable marker gene, and/or an antibiotics resistance gene. The polypeptide of interest may in particular be CD39 or a variant thereof, especially CD39 or a variant thereof as described herein.

In certain embodiments, the vector nucleic acid further comprises a coding sequence encoding CD39 or a variant thereof, especially CD39 or a variant thereof as described herein. The coding sequence encoding CD39 or a variant thereof may be present within the same expression cassette as the template sequence for the pri-miRNA, or may be present within a further expression cassette present within the vector nucleic acid.

In certain embodiments, the vector nucleic acid does not comprise a coding sequence encoding a polypeptide of interest. In specific embodiments where the vector nucleic acid does not comprise a coding sequence encoding the polypeptide of interest, the expression cassette according to the fourth aspect of the invention does not comprise a coding sequence for a polypeptide. In these embodiments, the vector nucleic acid in particular comprises a further expression cassette comprising a selectable marker gene. In alternative embodiments where the vector nucleic acid does not comprise a coding sequence encoding a polypeptide of interest, the expression cassette according to the fourth aspect of the invention comprises a coding sequence which encodes a selectable marker.

In certain embodiments, the vector nucleic acid comprises a coding sequence encoding a polypeptide of interest. The coding sequence encoding the polypeptide of interest may be present within the expressing cassette according to the fourth aspect of the invention or may be present in a further expression cassette. In specific embodiments, the vector nucleic acid comprises at least two expression cassettes, a first expression cassette for expression of the polypeptide of interest and a second expression cassette for the expression of a selectable marker, with either the first or the second expression cassette being an expressing cassette according to the fourth aspect of the invention. In alternative embodiments, the vector nucleic acid comprises at least three expression cassettes, a first expression cassette being an expressing cassette according to the fourth aspect of the invention, a second expression cassette for expression of the polypeptide of interest, and a third expression cassette for the expression of a selectable marker. The polypeptide of interest may in particular be CD39 or a variant thereof, especially CD39 or a variant thereof as described herein.

In certain embodiments, two or more of the expression cassettes of the vector nucleic acid are expression cassettes according to the fourth aspect of the invention. These expression cassettes may each comprise template sequences for the same or different pri-miRNAs, in particular for different pri-miRNAs. The miRNAs produced from the pri-miRNAs may in particular all target CCL2, but bind to different parts of the mRNA or pre-mRNA of CCL2.

### 5. Methods for Producing a CHO Cell

In a sixth aspect, the present invention provides a method for producing a CHO cell which is capable of expressing CD39 or a variant thereof, comprising introducing a vector nucleic acid according to the fifth aspect into a CHO cell. The vector nucleic acid may additionally comprise a coding sequence encoding CD39 or a variant thereof, or another nucleic acid comprising a coding sequence encoding CD39 or a variant thereof is present in or introduced into the CHO cell.

Thus, in certain embodiments the method for producing a CHO cell comprises the step of introducing a vector nucleic acid according to the fifth aspect into a CHO cell, wherein the vector nucleic acid comprises a coding sequence for CD39 or a variant thereof, either within the expression cassette which expresses the miRNA targeting CCL2, or within a further expression cassette. In alternative embodiments, the method for producing a CHO cell comprises the step of introducing a vector nucleic acid according to the fifth aspect into a CHO cell, wherein the vector nucleic acid does not comprise a coding sequence for CD39 or a variant thereof, and introducing a further vector nucleic acid suitable for recombinant expression of CD39 or a variant thereof into the CHO cell, wherein the different vector nucleic acids may be introduced into the CHO cell simultaneously or consecutively, in any order. In even further embodiments, the method for producing a CHO cell comprises the steps of (a) providing a CHO cell which is capable of expressing CD39 or a variant thereof, and (b) introducing a vector nucleic acid according to the fifth aspect into the CHO cell. In these embodiments, the vector nucleic acid according to the fifth aspect preferably does not comprise a coding sequence for CD39 or a variant thereof.

The CD39 or the variant thereof in particular is a CD39 or a variant thereof as described herein.

The vector nucleic acid is artificially introduced into the CHO cell. In particular, the vector nucleic acid is introduced by transfection. Transfection in this respect may be transient or stable, and especially stable transfection is used. Hence, in certain embodiments the produced CHO cell comprises the expression cassette according to the fifth aspect stably integrated into its genome.

In a further aspect, the present invention provides the use of the expression cassette according to the fourth aspect or the vector nucleic acid according to the fifth aspect or the CHO cell according to the sixth aspect for the production of CD39 or a variant thereof. The features, embodiments and examples of the method for producing CD39 or a variant thereof described herein likewise apply to this use.

The present invention further provides the use of the expression cassette according to the fourth aspect or the vector nucleic acid according to the fifth aspect for improving production of CD39 or a variant thereof by a CHO cell, including introducing the expression cassette or vector nucleic acid into a CHO cell capable of producing CD39 or a variant thereof. In certain embodiments, the vector nucleic acid introduced into the CHO cell does not comprise a coding sequence for CD39 or a variant thereof. Improving production of CD39 or a variant thereof in particular includes increasing the purity of CD39 or the variant thereof. The features, embodiments and examples of the method of improving production of CD39 or a variant thereof in a CHO cell described herein likewise apply to this use.

### 6. Compositions Comprising CD39 or a Variant Thereof

In a seventh aspect, the present invention provides a composition comprising CD39 or a variant thereof and CCL2, wherein the composition is obtained by production using CHO cells according to the second aspect, and wherein:
(i) the amount of CCL2 in the composition is at least 10-times lower compared to the same composition obtained by production using CHO cells which were not engineered to reduce the production of CCL2 in the CHO cells; and/or
(ii) the composition comprises CCL2 in a concentration of 100 ppm or lower, preferably 25 ppm or lower, more preferably 10 ppm or lower.

In certain embodiments, the composition is obtained by step (c1) of the method of producing CD39 or a variant thereof according to the first aspect of the invention. In these embodiments, the composition in particular may be a cell culture supernatant or a cell-free bulk harvest of a cell culture. The composition according to these embodiments in particular may comprise CCL2 in a concentration of 100 ppm or lower, preferably 25 ppm or lower, more preferably 10 ppm or lower.

In further embodiments, the composition is obtained by step (c4) of the method of producing CD39 or a variant thereof according to the first aspect of the invention. In these embodiments, the composition in particular may be a purified composition essentially free of proteins other than CD39 or a variant thereof. A composition essentially free of proteins other than CD39 or a variant thereof in particular comprises 5% or less, preferably 2% or less, more preferably 1% or less of proteins other than CD39 or a variant thereof relative to the entire amount of protein in the composition. The composition according to these embodiments in particular may comprise CCL2 in a concentration of 25 ppm or lower, preferably 10 ppm or lower, more preferably 5 ppm or lower.

In further embodiments, the composition is obtained by step (d) of the method of producing CD39 or a variant thereof according to the first aspect of the invention. In these embodiments, the composition in particular may be a drug product composition suitable for use in the treatment of patients, especially human patients. The composition according to these embodiments in particular may comprise CCL2 in a concentration of 25 ppm or lower, preferably 10 ppm or lower, more preferably 5 ppm or lower.

### 7. Specific Embodiments

In the following, specific embodiments of the present invention are described. These embodiments can be combined with the further embodiments, features and examples described herein.

Embodiment 1. A method of producing CD39 or a variant thereof, comprising the steps of:
(a) providing CHO cells capable of producing CD39 or a variant thereof;
(b) cultivating the CHO cells in a cell culture under conditions which allow for the production of CD39 or the variant thereof;
(c) obtaining CD39 or the variant thereof from the cell culture; and
(d) optionally processing CD39 or the variant thereof;
wherein the CHO cells are engineered to reduce the production of CCL2 in the CHO cells.

Embodiment 2. The method according to embodiment 1, wherein production of CCL2 in the CHO cells is reduced by knockdown or knockout of CCL2 expression or degradation of CCL2 protein.

Embodiment 3. The method according to embodiment 1 or 2, wherein the CHO cells are engineered to produce a miRNA targeting the endogenous CCL2 of the CHO cells.

Embodiment 4. The method according to embodiment 3, wherein the miRNA targeting CCL2 has a nucleotide sequence selected from the group consisting of SEQ ID NOs: 26 and 28.

Embodiment 5. The method according to embodiment 3 or 4, wherein the CHO cells comprise an expression cassette for production of the miRNA targeting CCL2.

Embodiment 6. The method according to embodiment 5, wherein the expression cassette comprises an intronic sequence comprising a template sequence for a pri-miRNA, wherein the pri-miRNA is suitable to be processed in the CHO cell to form the miRNA targeting CCL2.

Embodiment 7. The method according to embodiment 6, wherein the pri-miRNA comprises, from 5′ to 3′,
(i) a 5′ miRNA scaffold stem, optionally comprising the nucleotide sequence of SEQ ID NO: 1-4,
(ii) a passenger strand, having a nucleotide sequence complementary to the sequence of the miRNA, optionally comprising one or two mismatches,
(iii) a miRNA scaffold loop, optionally comprising the nucleotide sequence of SEQ ID NO: 8,
(iv) a guide strand, having the nucleotide sequence of the miRNA, and
(v) a 3′ miRNA scaffold stem, optionally comprising the nucleotide sequence of SEQ ID NO: 11-14;
   wherein the positions of the passenger strand and guide strand may be switched, and wherein optionally the passenger strand has the nucleotide sequence of SEQ ID NO: 25 and the guide strand has the nucleotide sequence of SEQ ID NO: 26, or the passenger strand has the nucleotide sequence of SEQ ID NO: 27 and the guide strand has the nucleotide sequence of SEQ ID NO: 28.

Embodiment 8. The method according to any one of embodiments 5 to 7, wherein the expression cassette comprises two or more template sequences for pri-miRNAs, wherein the pri-miRNAs are suitable to be processed in the CHO cell to form miRNAs targeting CCL2, wherein the miRNAs processed from the pri-miRNAs bind to different parts of the RNA, especially the mRNA or pre-mRNA, of CCL2.

Embodiment 9. The method according to embodiment 8, wherein the different miRNAs targeting CCL2 have nucleotide sequences selected from the group consisting of SEQ ID NOs: 26 and 28.

Embodiment 10. A method of producing CD39 or a variant thereof, comprising the steps of:
(a) providing CHO cells capable of producing CD39 or a variant thereof;
(b) cultivating the CHO cells in a cell culture under conditions which allow for the production of CD39 or the variant thereof;
(c) obtaining CD39 or the variant thereof from the cell culture; and
(d) optionally processing CD39 or the variant thereof.

Embodiment 11. The method according to any one of embodiments 1 to 10, wherein step (c) comprises performing anion exchange chromatography and/or hydrophobic interaction chromatography.

Embodiment 12. The method according to any one of embodiments 1 to 11, wherein step (c) comprises:
(c1) separating the CHO cells from cell culture supernatant containing CD39 or the variant thereof;
(c2) separating CD39 or the variant thereof from the cell culture supernatant using anion exchange chromatography;
(c3) further purifying CD39 or the variant thereof using hydrophobic interaction chromatography; and
(c4) further purifying CD39 or the variant thereof using anion exchange chromatography.

Embodiment 13. The method according to any one of embodiments 1 to 12, wherein step (d) comprises providing a pharmaceutical formulation comprising CD39 or the variant thereof.

Embodiment 14. The method according to any one of embodiments 1 to 13, wherein the method is for producing a variant of CD39 which has one or more of the following characteristics:
(i) it is a soluble variant lacking the membrane anchors of human CD39;
(ii) it has N terminal, C terminal and central deletions compared to human CD39;
(iii) it has an N terminal deletion of 30 to 50 amino acids, a C terminal deletion of 20 to 40 amino acids, and a central deletion of 10 to 15 amino acids compared to human CD39;
(iv) it comprise up to 5 point mutations compared to human CD39;
(v) it has an amino acid sequence selected from the group consisting of SEQ ID NOs: 29-49.

Embodiment 15. The method according to any one of embodiments 1 to 14, wherein the method is for producing a variant of CD39 comprising the amino acid sequence of SEQ ID NO: 49.

Embodiment 16. The method according to any one of embodiments 1 to 15, wherein the method is for producing a variant of CD39 consisting of the amino acid sequence of SEQ ID NO: 49.

Embodiment 17. The method according to any one of embodiments 1 to 16, wherein CD39 or the variant thereof obtained after step (c) is in a composition which comprises CCL2 in a concentration of 100 ppm or lower, preferably 25 ppm or lower, more preferably 10 ppm or lower.

Embodiment 18. The method according to any one of embodiment 12, wherein CD39 or the variant thereof obtained after step (c1) is in a composition which comprises CCL2 in a concentration of 100 ppm or lower, preferably 25 ppm or lower, more preferably 10 ppm or lower.

Embodiment 19. The method according to any one of embodiment 12, wherein CD39 or the variant thereof obtained after step (c4) is in a composition which comprises CCL2 in a concentration of 25 ppm or lower, preferably 10 ppm or lower, more preferably 5 ppm or lower.

Embodiment 20. The method according to any one of embodiments 1 to 19, wherein CD39 or the variant thereof obtained after step (d) is in a composition which comprises CCL2 in a concentration of 25 ppm or lower, preferably 10 ppm or lower, more preferably 5 ppm or lower.

Embodiment 21. The method according to any one of embodiments 1 to 20, wherein CD39 or the variant thereof obtained after step (c) is in a composition which comprises CCL2 in a concentration which is at least 10-times lower, preferably 25-times lower, more preferably 100-times lower, compared to a composition obtained with the same method after step (c) using CHO cells which are not engineered to reduce the production of CCL2 in the CHO cells.

Embodiment 22. The method according to any one of embodiments 1 to 20, wherein CD39 or the variant thereof obtained after step (c1) is in a composition which comprises CCL2 in a concentration which is at least 10-times lower, preferably 25-times lower, more preferably 100-times lower, compared to a composition obtained with the same method after step (c1) using CHO cells which are not engineered to reduce the production of CCL2 in the CHO cells.

Embodiment 23. The method according to any one of embodiments 1 to 20, wherein CD39 or the variant thereof obtained after step (c4) is in a composition which comprises CCL2 in a concentration which is at least 10-times lower, preferably 25-times lower, more preferably 100-times lower, compared to a composition obtained with the same method after step (c4) using CHO cells which are not engineered to reduce the production of CCL2 in the CHO cells. 20

Embodiment 24. The method according to any one of embodiments 1 to 20, wherein CD39 or the variant thereof obtained after step (d) is in a composition which comprises CCL2 in a concentration which is at least 10-times lower, preferably 25-times lower, more preferably 100-times lower, compared to a composition obtained with the same method after step (d) using CHO cells which are not engineered to reduce the production of CCL2 in the CHO cells.

Embodiment 25. A CHO cell which is capable of producing CD39 or a variant thereof and which is engineered to reduce the production of CCL2 in the CHO cell.

Embodiment 26. The cell according to embodiment 25, wherein production of CCL2 is reduced by knockdown or knockout of CCL2 expression or degradation of CCL2 protein.

Embodiment 27. The cell according to embodiment 25 or 26, which is engineered to produce a miRNA targeting the endogenous CCL2 of the CHO cells.

Embodiment 28. The cell according to embodiment 27, wherein the miRNA targeting CCL2 has a nucleotide sequence selected from the group consisting of SEQ ID NOs: 26 and 28.

Embodiment 29. The cell according to embodiment 27 or 28, comprising an expression cassette for production of the miRNA targeting CCL2.

Embodiment 30. The cell according to embodiment 29, wherein the expression cassette comprises an intronic sequence comprising a template sequence for a pri-miRNA, wherein the pri-miRNA is suitable to be processed in the CHO cell to form the miRNA targeting CCL2.

Embodiment 31. The cell according to embodiment 30, wherein the pri-miRNA comprises, from 5′ to 3′,
(i) a 5′ miRNA scaffold stem, optionally comprising the nucleotide sequence of SEQ ID NO: 1-4,
(ii) a passenger strand, having a nucleotide sequence complementary to the sequence of the miRNA, optionally comprising one or two mismatches,
(iii) a miRNA scaffold loop, optionally comprising the nucleotide sequence of SEQ ID NO: 8,
(iv) a guide strand, having the nucleotide sequence of the miRNA, and
(v) a 3′ miRNA scaffold stem, optionally comprising the nucleotide sequence of SEQ ID NO: 11-14;
   wherein the positions of the passenger strand and guide strand may be switched, and wherein optionally the passenger strand has the nucleotide sequence of SEQ ID NO: 25 and the guide strand has the nucleotide sequence of SEQ ID NO: 26, or the passenger strand has the nucleotide sequence of SEQ ID NO: 27 and the guide strand has the nucleotide sequence of SEQ ID NO: 28.

Embodiment 32. The cell according to any one of embodiments 29 to 31, wherein the expression cassette comprises two or more template sequences for pri-miRNAs, wherein the pri-miRNAs are suitable to be processed in the CHO cell to form miRNAs targeting CCL2, wherein the miRNAs processed from the pri-miRNAs bind to different parts of the RNA, especially the mRNA or pre-mRNA, of CCL2.

Embodiment 33. The cell according to embodiment 32, wherein the different miRNAs targeting CCL2 have nucleotide sequences selected from the group consisting of SEQ ID NOs: 26 and 28.

Embodiment 34. The cell according to any one of embodiments 25 to 33, capable of producing a variant of CD39 which has one or more of the following characteristics:
(i) it is a soluble variant lacking the membrane anchors of human CD39;
(ii) it has N terminal, C terminal and central deletions compared to human CD39;
(iii) it has an N terminal deletion of 30 to 50 amino acids, a C terminal deletion of 20 to 40 amino acids, and a central deletion of 10 to 15 amino acids compared to human CD39;
(iv) it comprise up to 5 point mutations compared to human CD39;
(v) it has an amino acid sequence selected from the group consisting of SEQ ID NOs: 29-49.

Embodiment 35. The cell according to any one of embodiments 25 to 34, capable of producing a variant of CD39 comprising the amino acid sequence of SEQ ID NO: 49.

Embodiment 36. The cell according to any one of embodiments 25 to 35, capable of producing a variant of CD39 consisting of the amino acid sequence of SEQ ID NO: 49. Embodiment 37. A method of improving production of CD39 or a variant thereof in a CHO cell, comprising the steps of
(a-i) providing a CHO cell capable of producing CD39 or a variant thereof;
(a-ii) engineering the CHO cell so as to reduce the production of CCL2 in the CHO cell.

Embodiment 38. The method according to embodiment 37, wherein the CHO cell is engineered in step (a-ii) by knockdown or knockout of CCL2 expression or inducing degradation of CCL2 protein in the CHO cell.

Embodiment 39. The method according to embodiment 37 or 38, wherein the CHO cell is engineered in step (a-ii) to produce a miRNA targeting the endogenous CCL2 of the CHO cells.

Embodiment 40. The method according to embodiment 39, wherein the miRNA targeting CCL2 has a nucleotide sequence selected from the group consisting of SEQ ID NOs: 26 and 28.

Embodiment 41. The method according to embodiment 39 or 40, wherein in step (a-ii) an expression cassette for production of the miRNA targeting CCL2 is introduced into the CHO cell.

Embodiment 42. The method according to embodiment 41, wherein the expression cassette comprises an intronic sequence comprising a template sequence for a pri-miRNA, wherein the pri-miRNA is suitable to be processed in the CHO cell to form the miRNA targeting CCL2.

Embodiment 43. The method according to embodiment 42, wherein the pri-miRNA comprises, from 5′ to 3′,
(i) a 5′ miRNA scaffold stem, optionally comprising the nucleotide sequence of SEQ ID NO: 1-4,
(ii) a passenger strand, having a nucleotide sequence complementary to the sequence of the miRNA, optionally comprising one or two mismatches,
(iii) a miRNA scaffold loop, optionally comprising the nucleotide sequence of SEQ ID NO: 8,
(iv) a guide strand, having the nucleotide sequence of the miRNA, and
(v) a 3′ miRNA scaffold stem, optionally comprising the nucleotide sequence of SEQ ID NO: 11-14;
   wherein the positions of the passenger strand and guide strand may be switched, and wherein optionally the passenger strand has the nucleotide sequence of SEQ ID NO: 25 and the guide strand has the nucleotide sequence of SEQ ID NO: 26, or the passenger strand has the nucleotide sequence of SEQ ID NO: 27 and the guide strand has the nucleotide sequence of SEQ ID NO: 28.

Embodiment 44. The method according to any one of embodiments 41 to 43, wherein the expression cassette comprises two or more template sequences for pri-miRNAs, wherein the pri-miRNAs are suitable to be processed in the CHO cell to form miRNAs targeting CCL2, wherein the miRNAs processed from the pri-miRNAs bind to different parts of the RNA, especially the mRNA or pre-mRNA, of CCL2.

Embodiment 45. The method according to embodiment 44, wherein the different miRNAs targeting CCL2 have nucleotide sequences selected from the group consisting of SEQ ID NOs: 26 and 28.

Embodiment 46. The method according to any one of embodiments 41 to 45, wherein step (a-ii) includes introducing a vector nucleic acid into the CHO cell, wherein the vector nucleic acid comprises the expression cassette for expression of the miRNA targeting CCL2.

Embodiment 47. The method according to any one of embodiments 37 to 46, wherein engineering the CHO cell reduces the level of CCL2 mRNA in the CHO cell by at least 5-fold, preferably at least 10-fold, more preferably at least 25-fold, compared to the same CHO cell prior to step (a-ii).

Embodiment 48. The method according to any one of embodiments 37 to 47, wherein engineering the CHO cell reduces the level of CCL2 protein in the CHO cell by at least 5-fold, preferably at least 10-fold, more preferably at least 25-fold, compared to the same CHO cell prior to step (a-ii).

Embodiment 49. The method according to any one of embodiments 37 to 48, wherein the method further comprises the steps of:
(b) cultivating the CHO cell obtained in step (a-ii) in a cell culture under conditions which allow for proliferation of the CHO cell and simultaneous and/or subsequent production of CD39 or the variant thereof;
(c) obtaining said CD39 or the variant thereof from the cell culture; and
(d) optionally processing CD39 or the variant thereof.

Embodiment 50. The method according to embodiment 49, wherein step (c) comprises performing anion exchange chromatography and/or hydrophobic interaction chromatography.

Embodiment 51. The method according to embodiment 49 or 50, wherein step (c) comprises
(c1) separating the CHO cells from cell culture supernatant containing CD39 or the variant thereof;
(c2) separating CD39 or the variant thereof from the cell culture supernatant using anion exchange chromatography;
(c3) further purifying CD39 or the variant thereof using hydrophobic interaction chromatography; and
(c4) further purifying CD39 or the variant thereof using anion exchange chromatography.

Embodiment 52. The method according to any one of embodiments 49 to 51, wherein step (d) comprises providing a pharmaceutical formulation comprising CD39 or the variant thereof.

Embodiment 53. The method according to any one of embodiments 49 to 52, wherein CD39 or the variant thereof obtained after step (c) is in a composition which comprises CCL2 in a concentration of 100 ppm or lower, preferably 25 ppm or lower, more preferably 10 ppm or lower.

Embodiment 54. The method according to any one of embodiments 51 to 53, wherein CD39 or the variant thereof obtained after step (c1) is in a composition which comprises CCL2 in a concentration of 100 ppm or lower, preferably 25 ppm or lower, more preferably 10 ppm or lower.

Embodiment 55. The method according to any one of embodiments 51 to 54, wherein CD39 or the variant thereof obtained after step (c4) is in a composition which comprises CCL2 in a concentration of 25 ppm or lower, preferably 10 ppm or lower, more preferably 5 ppm or lower.

Embodiment 56. The method according to any one of embodiments 49 to 55, wherein CD39 or the variant thereof obtained after step (d) is in a composition which comprises CCL2 in a concentration of 25 ppm or lower, preferably 10 ppm or lower, more preferably 5 ppm or lower.

Embodiment 57. The method according to any one of embodiments 49 to 56, wherein CD39 or the variant thereof obtained after step (c) is in a composition which comprises CCL2 in a concentration which is at least 10-times lower, preferably 25-times lower, more preferably 100-times lower, compared to a composition obtained with the same method after step (c) using a CHO cell which is not engineered to reduce the production of CCL2 in the CHO cell.

Embodiment 58. The method according to any one of embodiments 51 to 57, wherein CD39 or the variant thereof obtained after step (c1) is in a composition which comprises CCL2 in a concentration which is at least 10-times lower, preferably 25-times lower, more preferably 100-times lower, compared to a composition obtained with the same method after step (c1) using CHO cells which are not engineered to reduce the production of CCL2 in the CHO cells.

Embodiment 59. The method according to any one of embodiments 51 to 58, wherein CD39 or the variant thereof obtained after step (c4) is in a composition which comprises CCL2 in a concentration which is at least 10-times lower, preferably 25-times lower, more preferably 100-times lower, compared to a composition obtained with the same method after step (c4) using CHO cells which are not engineered to reduce the production of CCL2 in the CHO cells.

Embodiment 60. The method according to any one of embodiments 49 to 59, wherein CD39 or the variant thereof obtained after step (d) is in a composition which comprises CCL2 in a concentration which is at least 10-times lower, preferably 25-times lower, more preferably 100-times lower, compared to a composition obtained with the same method after step (d) using CHO cells which are not engineered to reduce the production of CCL2 in the CHO cells.

Embodiment 61. The method according to any one of embodiments 37 to 60, wherein the method is for improving production of a variant of CD39 which has one or more of the following characteristics:
(i) it is a soluble variant lacking the membrane anchors of human CD39;
(ii) it has N terminal, C terminal and central deletions compared to human CD39;
(iii) it has an N terminal deletion of 30 to 50 amino acids, a C terminal deletion of 20 to 40 amino acids, and a central deletion of 10 to 15 amino acids compared to human CD39;
(iv) it comprise up to 5 point mutations compared to human CD39;
(v) it has an amino acid sequence selected from the group consisting of SEQ ID NOs: 29-49.

Embodiment 62. The method according to any one of embodiments 37 to 61, wherein the method is for improving production of a variant of CD39 comprising the amino acid sequence of SEQ ID NO: 49.

Embodiment 63. The method according to any one of embodiments 37 to 62, wherein the method is for improving production of a variant of CD39 consisting of the amino acid sequence of SEQ ID NO: 49.

Embodiment 64. An expression cassette for expression of a miRNA in a CHO cell, comprising a template sequence for a pri-miRNA, wherein the pri-miRNA is suitable to be processed in a CHO cell to form a miRNA targeting CCL2.

Embodiment 65. The expression cassette according to embodiment 64, wherein the miRNA targeting CCL2 has a nucleotide sequence selected from the group consisting of SEQ ID NOs: 26 and 28.

Embodiment 66. The expression cassette according to embodiment 64 or 65, wherein the template sequence for the pri-miRNA is present within an intronic sequence.

Embodiment 67. The expression cassette according to any one of embodiments 64 to 66, wherein the pri-miRNA comprises, from 5′ to 3′,
(i) a 5′ miRNA scaffold stem, optionally comprising the nucleotide sequence of SEQ ID NO: 1-4,
(ii) a passenger strand, having a nucleotide sequence complementary to the sequence of the miRNA, optionally comprising one or two mismatches,
(iii) a miRNA scaffold loop, optionally comprising the nucleotide sequence of SEQ ID NO: 8,
(iv) a guide strand, having the nucleotide sequence of the miRNA, and
(v) a 3′ miRNA scaffold stem, optionally comprising the nucleotide sequence of SEQ ID NO: 11-14;
   wherein the positions of the passenger strand and guide strand may be switched, and wherein optionally the passenger strand has the nucleotide sequence of SEQ ID NO: 25 and the guide strand has the nucleotide sequence of SEQ ID NO: 26, or the passenger strand has the nucleotide sequence of SEQ ID NO: 27 and the guide strand has the nucleotide sequence of SEQ ID NO: 28.

Embodiment 68. The expression cassette according to any one of embodiments 64 to 67, comprising two or more template sequences for pri-miRNAs, wherein the pri-miRNAs are suitable to be processed in a CHO cell to form miRNAs targeting CCL2, wherein the miRNAs processed from the pri-miRNAs bind to different parts of the RNA, especially the mRNA or pre-mRNA, of CCL2.

Embodiment 69. The expression cassette according to embodiment 68, wherein the different miRNAs targeting CCL2 have nucleotide sequences selected from the group consisting of SEQ ID NOs: 26 and 28.

Embodiment 70. A vector nucleic acid for transfection of a CHO cell, comprising the expression cassette according to any one of embodiments 64 to 69.

Embodiment 71. The vector nucleic acid according to embodiment 70, further comprising a coding sequence encoding CD39 or a variant thereof.

Embodiment 72. The vector nucleic acid according to embodiment 71, wherein the coding sequence encoding CD39 or a variant thereof is present within the same expression cassette as the template sequence for the pri-miRNA.

Embodiment 73. The vector nucleic acid according to embodiment 71, wherein the coding sequence encoding CD39 or a variant thereof is present within a further expression cassette present within the vector nucleic acid.

Embodiment 74. The vector nucleic acid according to any one of embodiments 71 to 73, wherein the coding sequence encodes a variant of CD39 which has one or more of the following characteristics:
(i) it is a soluble variant lacking the membrane anchors of human CD39;
(ii) it has N terminal, C terminal and central deletions compared to human CD39;
(iii) it has an N terminal deletion of 30 to 50 amino acids, a C terminal deletion of 20 to 40 amino acids, and a central deletion of 10 to 15 amino acids compared to human CD39;
(iv) it comprise up to 5 point mutations compared to human CD39;
(v) it has an amino acid sequence selected from the group consisting of SEQ ID NOs: 29-49.

Embodiment 75. The vector nucleic acid according to any one of embodiments 71 to 74, wherein the coding sequence encodes a variant of CD39 comprising the amino acid sequence of SEQ ID NO: 49.

Embodiment 76. The vector nucleic acid according to any one of embodiments 71 to 74, wherein the coding sequence encodes a variant of CD39 consisting of the amino acid sequence of SEQ ID NO: 49.

Embodiment 77. A method for producing a CHO cell which is capable of expressing CD39 or a variant thereof, comprising introducing a vector nucleic acid according to any one of embodiments 70 to 76 into a CHO cell.

Embodiment 78. The method according to embodiment 77; wherein the CHO cell does not comprise a coding sequence encoding CD39 or a variant thereof prior to introduction of the vector nucleic acid; and wherein a vector nucleic acid according to any one of embodiments 71 to 76 is introduced into the CHO cell.

Embodiment 79. The method according to embodiment 77; wherein a vector nucleic acid according to embodiment 70 which does not comprise a coding sequence encoding CD39 or a variant thereof is introduced into the CHO cell; and wherein the CHO cell comprises a coding sequence encoding CD39 or a variant thereof prior to introduction of the vector nucleic acid.

Embodiment 80. The method according to embodiment 77; wherein a vector nucleic acid according to embodiment 70 which does not comprise a coding sequence encoding CD39 or a variant thereof is introduced into the CHO cell; and wherein a further vector nucleic acid comprising a coding sequence encoding CD39 or a variant thereof is introduced into the CHO cell; wherein introduction of said two different vector nucleic acids is performed either concomitantly or subsequently, in any order.

Embodiment 81. The method according to any one of embodiments 77 to 80, wherein the CHO cell is capable of expressing a variant of CD39 which has one or more of the following characteristics:
(i) it is a soluble variant lacking the membrane anchors of human CD39;
(ii) it has N terminal, C terminal and central deletions compared to human CD39;
(iii) it has an N terminal deletion of 30 to 50 amino acids, a C terminal deletion of 20 to 40 amino acids, and a central deletion of 10 to 15 amino acids compared to human CD39;
(iv) it comprise up to 5 point mutations compared to human CD39;
(v) it has an amino acid sequence selected from the group consisting of SEQ ID NOs: 29-49.

Embodiment 82. The method according to any one of embodiments 77 to 81, wherein the CHO cell is capable of expressing a variant of CD39 comprising the amino acid sequence of SEQ ID NO: 49.

Embodiment 83. The method according to any one of embodiments 77 to 82, wherein the CHO cell is capable of expressing a variant of CD39 consisting of the amino acid sequence of SEQ ID NO: 49.

Embodiment 84. A composition comprising CD39 or a variant thereof and CCL2, wherein the composition is obtained by production using CHO cells according to any one of embodiments 25 to 36, and wherein the amount of CCL2 in the composition is at least 10-times lower, preferably 25-times lower, more preferably 100-times lower, compared to the same composition obtained by production using CHO cells which were not engineered to reduce the production of CCL2 in the CHO cells.

Embodiment 85. A composition comprising CD39 or a variant thereof and CCL2, wherein the composition is obtained by production using CHO cells, and wherein the composition comprises CCL2 in a concentration of 100 ppm or lower, preferably 25 ppm or lower, more preferably 10 ppm or lower.

Embodiment 86. The composition according to embodiment 84, being a composition according to embodiment 85.

Embodiment 87. The composition according to any one of embodiments 84 to 86, being obtained by steps (a), (b) and (c1) of the method according to embodiment 12.

Embodiment 88. The composition according to embodiment 87, being a cell culture supernatant or a cell-free bulk harvest of a cell culture.

Embodiment 89. The composition according to embodiment 87 or 88, wherein the composition comprises CCL2 in a concentration of 100 ppm or lower, preferably 25 ppm or lower, more preferably 10 ppm or lower.

Embodiment 90. The composition according to any one of embodiments 84 to 86, being obtained by steps (a), (b) and (c) of the method according to any one of embodiments 1 to 24.

Embodiment 91. The composition according to any one of embodiments 84 to 86, being obtained by steps (a), (b), (c1), (c2), (c3) and (c4) of the method according to embodiment 12.

Embodiment 92. The composition according to embodiment 90 or 91, being a purified composition essentially free of proteins other than CD39 or a variant thereof.

Embodiment 93. The composition according to any one of embodiments 84 to 86, being obtained by steps (a), (b), (c) and (d) of the method according to any one of embodiments 1 to 24.

Embodiment 94. The composition according to any one of embodiments 90 to 93, being a purified composition essentially free of proteins other than CD39 or a variant thereof.

Embodiment 95. The composition according to any one of embodiments 90 to 94, wherein the composition comprises 5% or less, preferably 2% or less, more preferably 1% or less of proteins other than CD39 or a variant thereof relative to the entire amount of protein in the composition.

Embodiment 96. The composition according to any one of embodiments 90 to 95, wherein the composition comprises CCL2 in a concentration of 25 ppm or lower, preferably 10 ppm or lower, more preferably 5 ppm or lower.

Embodiment 97. A CHO cell which is engineered to reduce the production of CCL2 in the CHO cell.

Embodiment 98. The cell according to embodiment 97, wherein production of CCL2 is reduced by knockdown or knockout of CCL2 expression or degradation of CCL2 protein.

Embodiment 99. The cell according to embodiment 97 or 98, which is engineered to produce a miRNA targeting the endogenous CCL2 of the CHO cells.

Embodiment 100. The cell according to embodiment 99, wherein the miRNA targeting CCL2 has a nucleotide sequence selected from the group consisting of SEQ ID NOs: 26 and 28.

Embodiment 101. The cell according to embodiment 99 or 100, comprising an expression cassette for production of the miRNA targeting CCL2.

Embodiment 102. The cell according to embodiment 101, wherein the expression cassette comprises an intronic sequence comprising a template sequence for a pri-miRNA, wherein the pri-miRNA is suitable to be processed in the CHO cell to form the miRNA targeting CCL2.

Embodiment 103. The cell according to embodiment 102, wherein the pri-miRNA comprises, from 5′ to 3′,
(i) a 5′ miRNA scaffold stem, optionally comprising the nucleotide sequence of SEQ ID NO: 1-4,
(ii) a passenger strand, having a nucleotide sequence complementary to the sequence of the miRNA, optionally comprising one or two mismatches,
(iii) a miRNA scaffold loop, optionally comprising the nucleotide sequence of SEQ ID NO: 8,
(iv) a guide strand, having the nucleotide sequence of the miRNA, and
(v) a 3′ miRNA scaffold stem, optionally comprising the nucleotide sequence of SEQ ID NO: 11-14;
   wherein the positions of the passenger strand and guide strand may be switched, and wherein optionally the passenger strand has the nucleotide sequence of SEQ ID NO: 25 and the guide strand has the nucleotide sequence of SEQ ID NO: 26, or the passenger strand has the nucleotide sequence of SEQ ID NO: 27 and the guide strand has the nucleotide sequence of SEQ ID NO: 28.

Embodiment 104. The cell according to any one of embodiments 101 to 103, wherein the expression cassette comprises two or more template sequences for pri-miRNAs, wherein the pri-miRNAs are suitable to be processed in the CHO cell to form miRNAs targeting CCL2, wherein the miRNAs processed from the pri-miRNAs bind to different parts of the RNA, especially the mRNA or pre-mRNA, of CCL2.

Embodiment 105. The cell according to embodiment 104, wherein the different miRNAs targeting CCL2 have nucleotide sequences selected from the group consisting of SEQ ID NOs: 26 and 28.

### FIGURES

FIG. 1 shows the production and purification process of CD39*.

FIG. 2 shows the titer of CD39* and CCL2 in the cell culture supernatant during cultivation of the host cells and production of CD39*. CCL2 levels were detected by CCL2 ELISA.

FIG. 3 shows the elution profile of CD39* and CCL2 from the anion exchange capture chromatography step. CCL2 levels were detected by CCL2 ELISA.

FIG. 4 shows cell line engineering and development strategy for the CCL2 knockdown approach.

FIG. 5 shows cell viabilities (dotted lines) and viable cell densities of the different host cells during an optimized fed-batch. CCL2 knockdown pools (CCL2_A and CCL2_B) show similar viable cell densities as compared to controls (CD39* cell line, control miRNA, and empty CHO cell line).

FIG. 6 shows CD39* titers in the different host cells. Titers of CCL2 KD pools are comparable to controls (optimized fed-batch).

FIG. 7 shows relative mRNA expression of CD39*, CCL2 and control protein. At day 10 of the optimized fed-batch samples were taken and RNA was purified. CD39*, CCL2 and control gene transcripts were quantified using qPCR. Both CCL2 KD pools show significantly lower CCL2 transcript levels as compared to controls.

FIG. 8 shows CCL2 protein levels quantified using an CCL2 ELISA. CCL2 KD pools reveal lower CCL2 protein levels in non-purified harvest at day 10 of an optimized fed-batch run.

FIG. 9 shows CD39* titers of CCL2 KD pools and single cell clones. CCL2_A-encoding pools were used for single cell cloning. 96 clones were inoculated into a 24 dwp standard fed-batch and CD39* titers were assessed. As expected, some clones show higher and others lower titers as compared to originating pools and the CD39* cell line controls.

FIG. 10 shows CCL2 mRNA levels of CCL2 KD single cell clones. CCL2_A-encoding pools were used for single cell cloning. 96 clones were inoculated into a 24 dwp standard fed-batch and CCL2 mRNA levels were quantified using qPCR. The majority of clones show significantly reduced levels of CCL2 transcripts as compared to the CD39* cell line control (normalized to 1).

FIG. 11 shows the characterization of the top 3 CCL2 KD clones as compared to the CD39* cell line. A: CD39* titers derived from 7 L bioreactors are significantly higher in the CCL2 KD clones. B: CCL2 mRNA levels at day 10 of standard fed-batch performed in 7 L bioreactors are greatly reduced compared to the CD39* cell line (normalized to 1). Clone B shows more than 100 fold reduction of IGP transcripts. C: CCL2 protein levels (measured by CCL2 ELISA) in non-purified harvests derived from 7 L bioreactors runs show that the top 3 clones have CCL2 levels below the LOQ (<2 ppm) as compared to the CD39* cell line. 2 ppm corresponds to 2 ng CCL2/mg CD39.

### EXAMPLES

### Example 1: Identification of CCL2 as Critical Impurity of a Human CD39 Variant Recombinantly Produced in CHO Cells

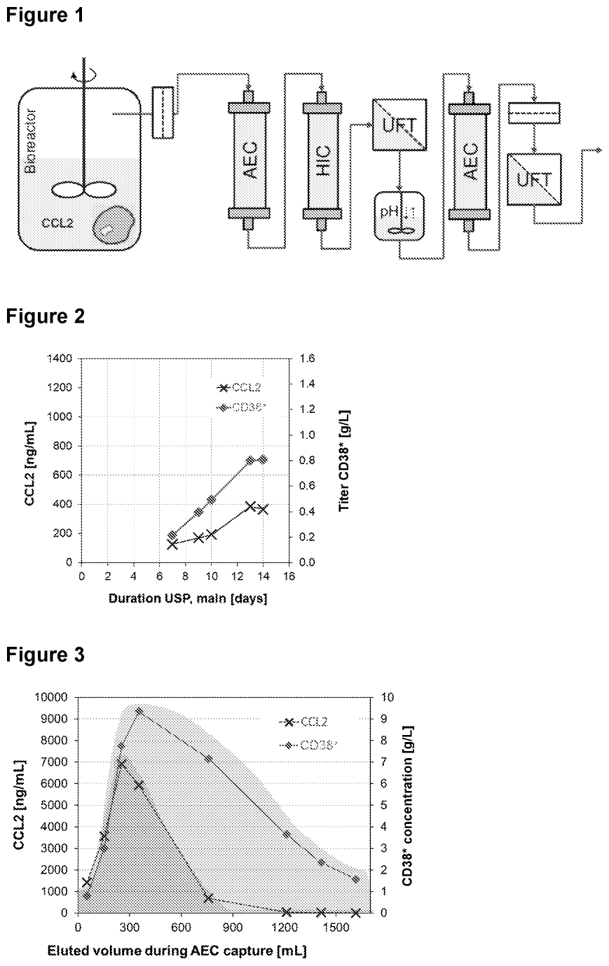

A variant of human CD39 having the amino acid sequence of SEQ ID NO: 49 (in the following “CD39*”) was produced by a CHO production cell line (“CD39* cell line”) and isolated from the cell culture using a standard purification process. The CD39* drug product purification process followed a 3-chromatography step process, which includes anion exchange, hydrophobic interaction and multimodal anion exchange chromatography, low pH virus inactivation, virus removal filtration and two ultrafiltration/diafiltration steps (FIG. 1).

Applying the purification process for CD39* showed depletion of host cell proteins (HCPs) using an ELISA assay specific for multiple HCPs. Analysis of the individual HCPs using an orthogonal LC-MS method revealed one prominent protein: CCL2 (CC-chemokine ligand 2; also known as MCP-1 (monocyte chemoattractant protein 1)), which was not detected by ELISA.

TABLE 1
Amount of CCL2 in the purified product

| | CCL2 [ppm] |
|---|---|
| Batch 1 | 366.9 |
| Batch 2 | 368.5 |
| Batch 3 | 410.8 |

It was further observed that CCL2 was not removed during the purification process as its concentration in the product remained nearly constant after each purification step.

A further analysis of the upstream process (USP) and downstream process (DSP) of CD39* production showed that formation of CCL2 in the cell culture increased with increasing cultivation time (FIG. 2), and that the elution profile of CCL2 in the AEC capture step overlapped significantly with the elution profile of CD39* (FIG. 3).

### Example 2: MiRNA Vector Design

The intronic-miRNA encoding vectors (pCMV04_A and pCMV04_B) were based on a standard vector (pCMV). The vector was modified by insertion of the intronic miRNA sequences into the cloning site of the CMV promoter-driven expression cassette. The sequence environment selected for the miRNA scaffold is similar to the human miR-30A and the miR-E molecules (e.g. Fellmann et al., 2011, Molecular Cell 41, 733-746) and is expected to lead to optimal processing of resulting miRNA sequences. However, the sequence was further modified: i) the EcoRI restriction site was replaced with BgIII restriction site, ii) the sequence downstream of the XhoI restriction site was replaced with a CHO-derived sequence and iii) additional miR-30A scaffold was added up- and downstream of the published sequence. Two different miRNA sequences targeting endogenous CCL2 of the CHO cells were tested: CCL2_A AIM (artificial intronic miRNA) comprising a guide strand having the nucleotide sequence of SEQ ID NO: 26 and a passenger strand having the nucleotide sequence of SEQ ID NO: 25, and CCL2_B AIM comprising a guide strand having the nucleotide sequence of SEQ ID NO: 28 and a passenger strand having the nucleotide sequence of SEQ ID NO: 27. In addition, a vector with an AIM targeting an unrelated endogenous protein of the CHO cells was used as control.

### Example 3: CCL2 Knockdown Cell Line Generation

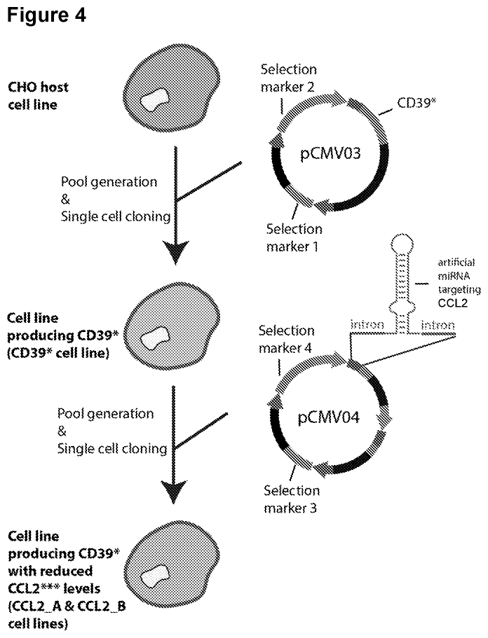

The strategy of CCL2 KD cell line generation is shown in FIG. 4. The CHO parental cell line was transfected with vector pCMV03 encoding a variant of human CD39 (in the following “CD39*”) and selection of pools were performed using MTX in low folate medium. The pools were going into single cell cloning and a monoclonal cell line expressing CD39* was selected, called CD39* cell line. Subsequently, the primary seed lot (PSL) of the CD39* cell line was used to transfect a vector encoding the artificial intronic miRNA targeting endogenous CCL2 and pools were generated using puromycine.

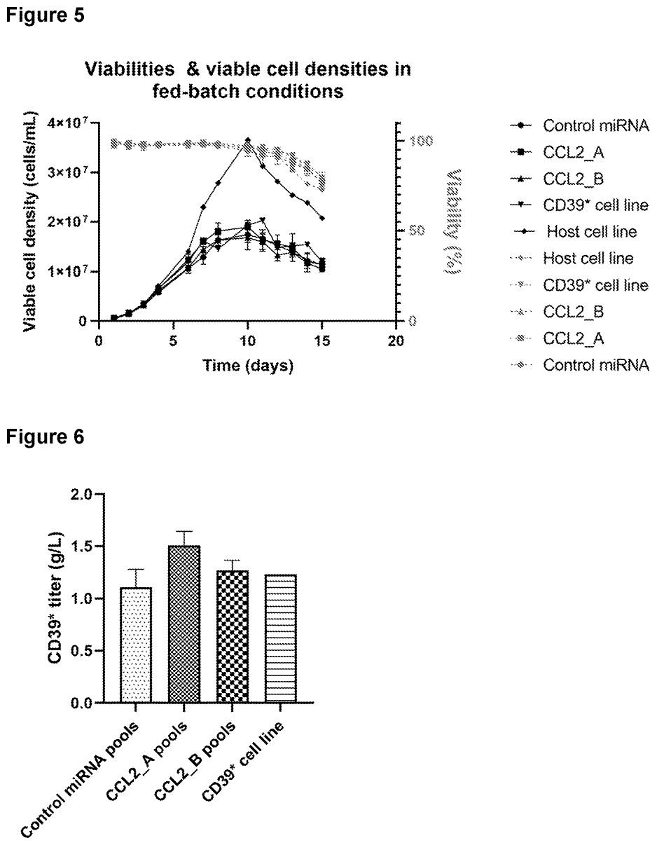

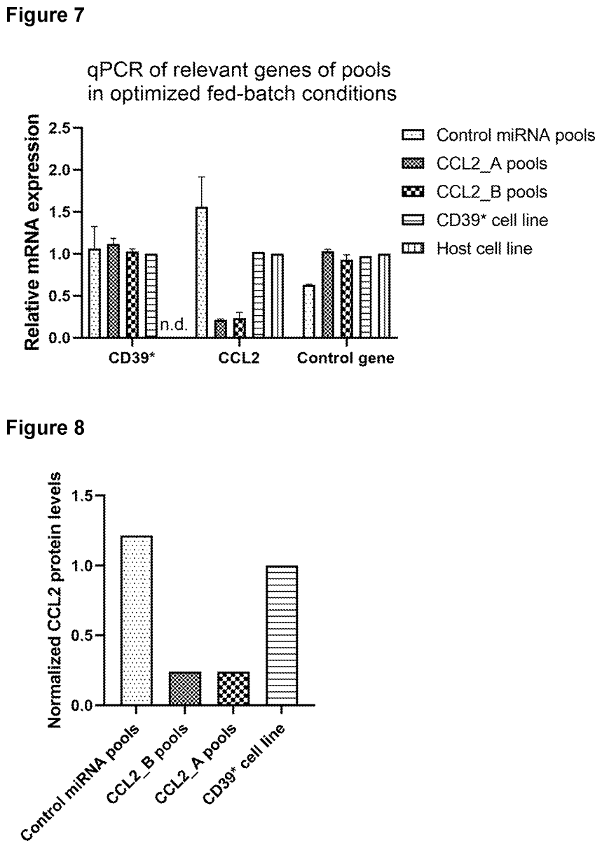

Two different miRNAs were generated targeting CHO CCL2 mRNA called CCL2_A and CCL2_B, both targeting the 3′UTR of the transcript. For pool generations, a miRNA targeting a different gene (control AIM), the parental CD39* cell line as well as the empty parental cell line (CHO) as controls were included. All samples (triplicate pool generations for the knockdown approaches) were inoculated into an optimized fed-batch run and cell growth, gene expression and CD39* titers were assessed at different days (FIGS. 5 to 7). Also, the CCL2 protein levels on harvest level were assessed using an CCL2 ELISA (FIG. 8):

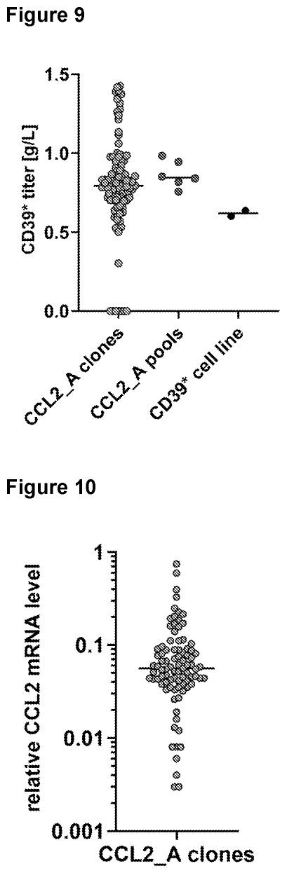

After pool generations and confirmation of an efficient CCL2 knockdown on mRNA level, the CCL2_A pools were selected for single cell cloning and 96 expanded clones were inoculated into a 24 dwp standard fed-batch to assess CCL2 knockdown efficiencies and CD39* productivities (FIGS. 9 and 10).

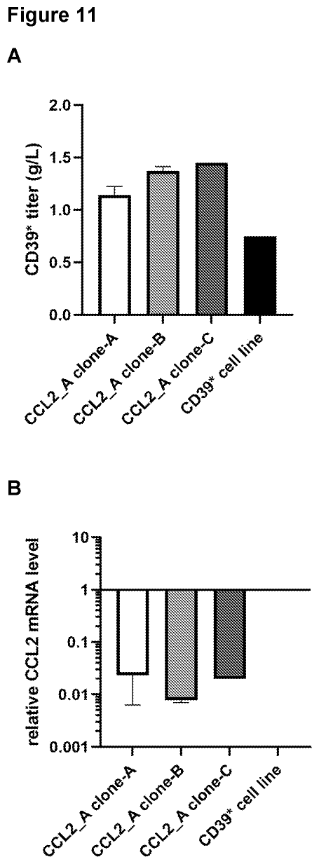

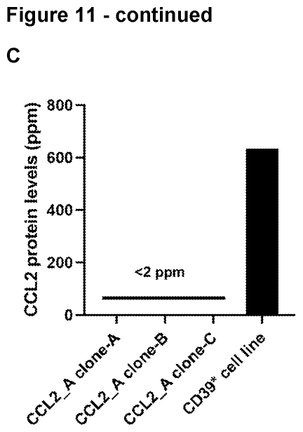

The top 30 clones were further characterized. Based on many parameters (USP, DSP, CCL2 data, CD39* protein characteristics) the top 3 clones were selected and inoculated into a 7 L bioreactor. CCL2 expression was significantly reduced in the CCL2 knockdown clones, resulting in an increased CD39* titer (FIG. 11).

TABLE 2
Properties of selected CCL2 AIM clones

| | | PVCD | | Copy | |
|---|---|---|---|---|---|
| | Viability | [10⁶ | CCL2 | number | CCL2 |
| Clone | EoC [%] | VC/mL] | mRNA* | miR-CCL2 | [ppm] |
| CCL2 AIM | 86.6 | 13.71 | 0.008 | 0.3 | <2 |
| clone A | | | | | |
| CCL2 AIM | 91.1 | 11.01 | 0.006 | 0.8 | <4 |
| clone B | | | | | |
| CCL2 AIM | 93.0 | 13.63 | 0.018 | 1.3 | <4 |
| clone C | | | | | |
| CD39* cell | 90.3 | 9.17 | 1.000 | 0.0 | 377.3 |
| line | | | | | |

*normalized against CD39* cell line

After harvesting the culture supernatant containing the CD39* protein, the concentration of CCL2 in the cell-free supernatant was determined.

TABLE 3
Amount of CCL2 in cell-free supernatant

| | CCL2 [ppm] |
|---|---|
| Batch 1 | 4.4 |
| Batch 2 | <4 |
| Batch 3 | <8 |

Subsequent application of downstream process including anion exchange chromatographic, HIC and MAC led to a CD39* composition (drug substance) with a CLL2 content below the limit of quantification (4 ppm):

TABLE 4
Amount of CCL2 in purified drug substance

| | CCL2 [ppm] |
|---|---|
| Batch 1 | <4 |
| Batch 2 | <4 |
| Batch 3 | <4 |

### Example 4: CCL2 Knockout Cell Line Generation

Gene-editing technologies represent alternative methods of the vectorized RNAi approach using artificial intronic miRNAs. A gene knockout on the DNA level which encodes the CCL2 gene leads to a complete functional deactivation due to loss of essential gene information. We generated single gRNA pairs for the targeting of Cas-Clover to the CCL2 gene on exons 1 and 2 (guide CCL2-01: SEQ ID NO: 51; guide CCL2-02: SEQ ID NO: 52; guide CCL2-03: SEQ ID NO: 53; guide CCL2-04: SEQ ID NO: 54). Cas-Clover, a dCas9 protein fused to the nuclease Clo51, can homodimerize at the CCL2 locus leading to insertion and deletion (indel) formation and eventually to frameshift mutations of the CCL2 gene. mRNA encoding Cas-Clover was transfected into MaKo cells seeded into 24-well plates one day prior transfection. After 6h, a sgRNA pair targeting the CCL2 gene was co-transfected to Cas-Clover-expressing cells. One day after transfection, the living cell population was single cell sorted using fluorescently-activated cell sorting (FACS). After a 16 days expansion period in 96-well plates, genomic DNA of clonal cells were isolated for NGS genotyping. PCR amplicons created with primers flanking the expected gRNA editing sites were sequenced as Nextera XT libraries on an Illumina's MiSeq. The sequencing reads were aligned to the CCL2 gene sequence to identify indels. Clone 048, which was transfected with the sgRNA pair guide_SA03 and guide_SA04, revealed two deletions (40 bp and 15 bp-13 bp) without detection of wildtype sequence confirming a homozygous CCL2 gene knockout in the CHO MaKo cell line.

### Example 5: Materials and Methods

The following materials and methods were used in Examples 1 to 4.

### 1. Expression Vector Construction

The vectors used in the examples consist of following elements: hCMV promoter/enhancer driving expression of the individual genes, polyadenylation signal (polyA), folic acid receptor, DHFR, puromycin and hygromycin resistance genes as selection markers, E. coli origin (ColE ori) of replication and the beta-lactamase gene for ampicillin (amp) resistance to enable amplification in bacteria. Different plasmid setups were evaluated and more details are provided within the figures.

### 2. Cell Lines, Cultivation, Transfection and Selection

CHO cell lines were cultivated in 24-deep well plates or shake flasks in a non-humidified shaker cabinet at 300 rpm (24 dwp) or 150 rpm (shake flasks), 10% CO₂ at 36.5° C. in suspension in proprietary, chemically defined culture media. Cell viabilities and growth rates were monitored by means of an automated system (ViCell, Beckman Coulter) or using an analytical flow cytometry (CytoFlex, Beckman Coulter). Cells were passaged 2-3 times per week into fresh medium and were maintained in logarithmic growth phase.

Linearized expression vectors were transfected by electroporation (Amaxa Nucleofection system, Lonza, Germany). The transfection reaction was performed in chemically defined cultivation medium, according to the manufactures instructions. The parental CHO cells used for transfection were in exponential growth phase with cell viabilities higher than 95%. Transfections were performed with 5× 10⁶ cells per transfection. Immediately, after transfection cells were transferred into shake flasks, containing chemically defined cultivation medium. Cell pools were incubated for 48 hours at 36.5° C. and 10% CO₂ before starting the selection process.

A selection procedure was carried out using the selection markers encoded by the individual expression vectors, as described above. The proteins FolR and DHFR are participating in the same molecular pathway; the FolR is transporting folic acid as well as the folate analogue MTX into the cell, the DHFR is converting it into vital precursors for purine and methionine synthesis. Combining them as selective principle, a particular strong selective regime can be taken to enrich for recombinant cells expressing both recombinant protein. Puromycin selection is driven by its inhibition of protein synthesis and vectors encoding the puromycin resistance marker gene enable cells to survive in presence of puroymycin.

48 h after transfection and growth under low folate conditions, additional selective pressure was applied by adding 10 nM MTX to the chemically defined cultivation medium. Alternatively, puromycin was used as selection agent. 48 h after transfection 0.003 mg/mL puromycin was added to the chemically defined cultivation medium. After pool recovery cells were frozen in culture medium, supplemented with 7.5% DMSO and cell pellets prepared.

### 3. Gene Expression Analysis by Quantitative Real-Time PCR

RNA extraction was performed using the Qiagen RNeasy Mini Kit according to the manufactures instructions. For real-time qPCR, cDNA was synthesized from 200 ng/μl diluted RNA using the High Capacity RNA-to-cDNA Master Mix (Applied Biosystems) and 10× diluted cDNAs were analyzed in triplicates using the QuantiFast SYBR Green PCR Kit (Qiagen) or TaqMan Primer/Probe system and TaqMan Mastermix (Applied Biosystems). As endogenous control for normalization GAPDH was amplified. Amplification and analysis was performed using the ABI PRISM® 7900HT Sequence Detection System. For calculation of relative quantities (RQ) of gene expression for sample comparison the comparative 2^{-ΔΔct} method was used and the data normalized.

### 4. Upstream Processing

Subsequent to selection, material was produced either in shake flask fed batch, 24-deep well plate cultures or ambr15 bioreactors. Fed batch cultures were inoculated with a cell seeding density of 4E5 vc/ml (addition of proprietary feed solutions starting on day 3 and cultivation temperature shift to 33° C. on day 5). During the cultivation in-process controls were performed to monitor the concentration of CD39*. Cell culture samples for RNA isolation were taken at day 10 of the process. The individual culture was cultivated over a period of 14 days. At the end of the cultivation process cells were separated from the culture supernatant by centrifugation and/or depth filtration followed by sterile filtration before further downstream processing.

### 5. CCL2 Protein Analysis by ELISA

The amount of Chinese hamster (CHO) host cell protein “CC-chemokine ligand 2” (CCL2) was determined using a sandwich ELISA. Samples were added to microtiter plates coated with anti-CCL2 antibody (capture antibody). Bound CCL2 is then quantified by incubation with biotinylated anti-CCL2 antibody (detection antibody), followed by streptavidin-peroxidase and tetramethylbenzidine (TMB) as substrate and measuring absorbance at 450 nm. The CCL2 levels in samples were calculated based on rat or CHO CCL2 standard, preferably CHO CCL2 standard.

### 6. CCL2 Determination by LC-MS

The amount of Chinese hamster (CHO) host cell protein “CC-chemokine ligand 2” (CCL2) was determined using LC-MS absolute quantification. CCL2 heavy labelled peptides derived from CCL2 sequence are spiked into tryptic digests of drug substance. Endogenous CCL2 protein is then quantified by comparing LC-MS intensities of endogenous CCL2 non-labelled peptides against LC-MS intensities of spiked CCL2 heavy labelled peptides.

The method demonstrated a linear detection of CCL2 heavy labelled peptides over a range between 33 ng/mg DS and 8100 ng/mg DS (R2>0.99). CCL2 quantification in CD39* was not influenced by the concentration of the heavy labelled peptide used in the experiment (99% agreement), which was confirmed using an alternative data analysis (peak area vs. MS intensities). The method demonstrated a precision of 9% CV. By conclusion, the method is considered suitable for quantifying CCL2 in CD39* drug substance.

SEQUENCE LISTING

| SEQ ID | | |
|---|---|---|
| NO | Description | Sequence |
| 1 | 5′ miRNA scaffold stem | GTGCTCGACTAGGGATAACAGGGTAATTGTTTGAATGAGGCTTCA |
| | | GTACTTTACAGAATCGTTGCCTGCACATCTTGGAAACACTTGCTG |
| | | GGATTACTTCAGCTCTTTAACCCAACAGAAGGCTCGAGAGAAAGC |
| | | ATATCTGTTGACAGTGAGCG |
| 2 | 5′ miRNA scaffold stem | GTGCTCGACTAGGGATAACAGGGTAATTGTTTGAATGAGGCTTCA |
| | | GTACTTTACAGAATCGTTGCCTGCACATCTTGGAAACACTTGCTG |
| | | GGATTACTTCAGCTCTTTAACCCAACAGAAGGCTCGAGGCTAGCG |
| | | CCGATATAACGGCGGAGAAAGCATATCTGTTGACAGTGAGCG |
| 3 | 5′ miRNA scaffold stem | GTGCTCGACTAGGGATAACAGGGTAATTGTTTGAATGAGGCTTCA |
| | | GTACTTTACAGAATCGTTGCCTGCACATCTTGGAAACACTTGCTG |
| | | GGATTACTTCACCTGTTTAACCCAACAGAAGGCTTAAGAGAAAGC |
| | | ATATCTGTTGACAGTGAGCG |
| 4 | 5′ miRNA scaffold stem | GTGCTCGACTAGGGATAACAGGGTAATTGTTTGAATGAGGCTTCA |
| | | GTACTTTACAGAATCGTTGCCTGCACATCTTGGAAACACTTGCTG |
| | | GGATTACTTCGACTTTTCTAACCAACAGAAGGCGTACGAGAAAGC |
| | | ATATCTGTTGACAGTGAGCG |
| 5 | 5′ miRNA scaffold stem | GTGCTCGACTAGGGATAACAGGGTAATTGTTTGAATGAGGCTTCA |
| | | GTACTTTACAGAATCGTTGCCTGCACATCTTGGAAACACTTGCTG |
| | | GGATTACTTCGACTTCTTAACCCAACAGAAGGCTCGAGAAGGTAT |
| | | ATTGCTGTTGACAGTGAGCG |
| 6 | 5′ miRNA scaffold stem | CTCGAGCCGGATCAACGCCCTAGGTTTATGTTTGGATGAACTGAC |
| | | ATCCGCGTATCCGTC |
| 7 | 5′ miRNA scaffold stem | CTCGAGCAGCCAGCTTTTTGCGAATCTCGACA |
| 8 | miRNA scaffold loop | TAGTGAAGCCACAGATGTA |
| 9 | miRNA scaffold loop | GTAGTGAAATATATATTAAAC |
| 10 | miRNA scaffold loop | TGTGTTTTTTTTGAA |
| 11 | 3′ miRNA scaffold stem | TGCCTACTGCCTCGGACTTCAAGGGGCTAAGATCTGGCAATTATC |
| | | TTGTTTACTAAAACTGAATACCTTGCTATCTCTTTGATACATTTT |
| | | TACAAAGCTGAATTAAAATGGTATAAATTAAATCACTTTTTT |
| 12 | 3′ miRNA scaffold stem | TGCCTACTGCCTCGGACTTCAAGGGACTAGTAGATCTCAATTATC |
| | | TTGTTTACTAAAACTGAATACCTTGCTATCTCTTTGATACATTTT |
| | | TACAAAGCTGAATTAAAATGGTATAAATTAAATCACTTTTTT |
| 13 | 3′ miRNA scaffold stem | TGCCTACTGCCTCGGACTTCAAGGGGCTAACCGGTGGCAATTATC |
| | | TTGTTTACTAAAACTGAATACCTTGCTATCTCTTTGATACATTTT |
| | | TACAAAGCTGAATTAAAATGGTATAAATTAAATCACTTTTTT |
| 14 | 3′ miRNA scaffold stem | TGCCTACTGCCTCGGACTTCAAGGGGCTATTCGAAGCAATTATCT |
| | | TGTTTACTAAAACTGAATACCTTGCTATCTCTTTGATACATTTTT |
| | | ACAAAGCTGAATTAAAATGGTATAAATTAAATCACTTTTTT |
| 15 | 3′ miRNA scaffold stem | TGCCTACTGCCTCGGACTTCAAGGGGCTAGAATTCGGCAATTATC |
| | | TTGTTTACTAAAACTGAATACCTTGCTATCTCTTTGATACATTTT |
| | | TACAAAGCTGAATTAAAATGGTATAAATTAAATCACTTTTTT |
| 16 | 3′ miRNA scaffold stem | TACGGTAACGCGGAATACGCAACTATTTTATCAATTTTTTGCGTC |
| | | GACAGACTC |
| 17 | 3′ miRNA scaffold stem | TCGCGATTCGCTTTTTCGTCTTTGAGATCT |
| 18 | synthetic intron with splice | GGAACGGTGCATTGGAACGCGGATTCCCCGTGCCAAGAGTGACGT |
| | donor | AAGTACCGCCT |
| 19 | synthetic intron with splice | ATAGAGTCTATAGGCCCACCCCCTTGGCTTCGTTAGAACGCGGCT |
| | acceptor | ACAATTAATACATAACCTTATGTATCATACACATACGATTTAGGT |
| | | GACACTATAGAATAACATCCACTTTGCCTTTCTCTCCACAGGTGT |
| | | CCACTCCCAGGTCCAACTGC |
| 20 | synthetic intron with splice | CAGTTCGAAGAGGTAAGT |
| | donor | |
| 21 | synthetic intron with splice | TACTAACTCTTCTTTTTTTTTTTCACAGGACCATCGATCGAA |
| | acceptor | |
| 22 | spacer sequence | CCGCCGATATAACGGCGG |
| 23 | Cricetulus griseus CCL2 nt | CTTCCACTTTTCCTCACTCACTGACGATGCTGCCCCTGGGGACCA |
| | | GGCTCCCAGCCTGACTCCACCCACTGGCTCACTATAAAGGCTGAG |
| | | GCAGAGCTGCCAGAAGAGCAGAGAGACTGAGGCCAACCCAGACCT |
| | | GCCTATTCTCTCCGAAGCCAGCTCTCTCTTCTTCCACCATGCAGT |
| | | TCTCCGCAAGAACGCTTCTGTGCCTGCTACTCACAGTTGCTGCCT |
| | | GTAGCATCTATGTGCTGGCCCAGCCAGACGCTGTTAACTCCCCCC |
| | | TCACCTGCTGCTACTCATTTACCGCCAAGAGGATCCCAGAGAAGA |
| | | GGCTGGAGAGCTACAAGAGAATCACCAGCAGCAAGTGTCCCAAAG |
| | | AAGCTGTGATTTTCATCACCAAGCTCAAGAGAGAGATCTGTGCTG |
| | | ACCCCAAGCAGGACTGGGTCCAGACATACACAAAAAAACTGGACC |
| | | AGAGCCAAGCTAAATCAGAAGCTGCAACTGTGTATAAAACAGCAC |
| | | CTTTGAATGCTAACTTGACCCATGAGTCTGCAGTTAATGCATCCA |
| | | CTACTGCCTTTCCCACCACAGACTTGAGAACTTCTGTAAGAGTGA |
| | | CCAGTATGACAGTGAACTAGTGTGACCTGGAATGTGATGCCTTAA |
| | | GTAATATTAACTTTATTTAACTTATTAATGTTTCAATATCCCCTT |
| | | CCCTGTATACTTGGATTTCTTAGATGCATGACGTGGTTTCATTGT |
| | | TTTCCCTCTGTGAACCCTGGTTCTCCACCCTAAATGTTATGAGAG |
| | | ATGACTTTTGTAAAGATGAGTGAAAAAAAAAACGTTGTGTTTGAG |
| | | ATCCCAAGGCATTGTTTAAAATATTATTATGGAAATGGATATTTC |
| | | TAATTATTAAGAGGAATAAATTATTTTTGTACATA |
| 24 | Cricetulus griseus CCL2 aa | MQFSARTLLCLLLTVAACSIYVLAQPDAVNSPLTCCYSFTAKRIP |
| | | EKRLESYKRITSSKCPKEAVIFITKLKREICADPKQDWVQTYTKK |
| | | LDQSQAKSEAATVYKTAPLNANLTHESAVNASTTAFPTTDLRTSV |
| | | RVTSMTVN |
| 25 | miRNA passenger strand | CGAAATGGATATTTCTAATTAT |
| | CCL2_A | |
| 26 | miRNA guide strand | ATAATTAGAAATATCCATTTCC |
| | CCL2_A | |
| 27 | miRNA passenger strand | AACCCTAAATGTTATGAGAGAT |
| | CCL2_B | |
| 28 | miRNA guide strand | ATCTCTCATAACATTTAGGGTG |
| | CCL2_B | |
| 29 | human CD39 variant | TQNKALPENVKYGIVLDAGSSHTSLYIYKWPAEKENDTGVVHQVE |
| | | ECRVKGPGISKFVQKVNEIGIYLTDCMERAREVIPRSQHQETPVY |
| | | LGATAGMRLLRMESEELADRVLDVVERSLSNYPFDFQGARIITGQ |
| | | EEGAYGWITINYLLGKFSQKNQETFGALDLGGASTQVTFVPQNQT |
| | | IESPDNALQFRLYGKDYNVYTHSFLCYGKDQALWQKLAKDIQVAS |
| | | NEILRDPCFHPGYKKVVNVSDLYKTPCTKRFEMTLPFQQFEIQGI |
| | | GNYQQCHQSILELFNTSYCPYSQCAFNGIFLPPLQGDFGAFSAFY |
| | | FVMKFLNLTSEKVSQEKVTEMMKKFCAQPWEEIKTSYAGVKEKYL |
| | | SEYCFSGTYILSLLLQGYHFTADSWEHIHFIGKIQGSDAGWTLGY |
| | | MLNLTNMIPAEQPLSTPLSHST |
| 30 | human CD39 variant | TQNKALPENVKYGIVLDAGSSHTSLYIYKWPAEKENDTGVVHQVE |
| | | ECRVKGPGISKFVQKVNEIGIYLTDCMERAMEVIPRSQHQETPVY |
| | | LGATAGMRLLRMESEELADRVLDVVERSLSNYPFDFQGARIITGQ |
| | | EEGAYGWITINYLLGKFSQKNQETFGALDLGGASTQVTFVPQNQT |
| | | IESPDNALQFRLYGKDYNVYTHSFLCYGKDQALWQKLAKDIQVAS |
| | | NEILRDPCFHPGYKKVVNVSDLYKTPCTKRFEMTLPFQQFEIQGI |
| | | GNYQQCHQSILELFNTSYCPYSQCAFNGIFLPPLQGDFGAFSAFY |
| | | FVMKFLNLTSEKVSQEKVTEMMKKFCAQPWEEIKTSYAGVKEKYL |
| | | SEYCFSGTYILSLLLQGYHFTADSWEHIHFIGKIQGSDAGWTLGY |
| | | MLNLTNMIPAEQPLSTPLSHST |
| 31 | human CD39 variant | TQNKALPENVKYGIVLDAGSSHTSLYIYKWPAEKENDTGVVHQVE |
| | | ECRVKGPGISKFVQKVNEIGIYLTDCMERAREVIPRSQHQETPVY |
| | | LGATAGMRLLRMESEELADRVLDVVERSLSNYPFDFQGARIITGQ |
| | | EEGAYGWITINYLLGKFSQKNQETFGALDLGGASTQVTFVPQNQT |
| | | IESPDNALQFRLYGKDYNVYTHSFLCYGKDQALWQKLAKDIQVAS |
| | | NEILRDPCFHPGYKKVVNVSDLYKTPCTKRFEMTLPFQQFEIQGI |
| | | GNYQQCHQSILELFNTSYCPYSQCAFNGIFLPPLQGDFGAFSAFY |
| | | SVMKFLNLTSEKVSQEKVTEMMKKFCAQPWEEIKTSYAGVKEKYL |
| | | SEYCFSGTYILSLLLQGYHFTADSWEHIHFIGKIQGSDAGWTLGY |
| | | MLNLTNMIPAEQPLSTPLSHST |
| 32 | human CD39 variant | TQNKALPENVKYGIVLDAGSSHTSLYIYKWPAEKENDTGVVHQVE |
| | | ECRVKGPGISKFVQKVNEIGIYLTDCMERAMEVIPRSQHQETPVY |
| | | LGATAGMRLLRMESEELADRVLDVVERSLSNYPFDFQGARIITGQ |
| | | EEGAYGWITINYLLGKFSQKNQETFGALDLGGASTQVTFVPQNQT |
| | | IESPDNALQFRLYGKDYNVYTHSFLCYGKDQALWQKLAKDIQVAS |
| | | NEILRDPCFHPGYKKVVNVSDLYKTPCTKRFEMTLPFQQFEIQGI |
| | | GNYQQCHQSILELFNTSYCPYSQCAFNGIFLPPLQGDFGAFSAFY |
| | | FVMKFLNLTSEKVSQEKVTEMMKKFCAQPWEEIKTSYAGVNEKYL |
| | | SEFCFSGTYILSLLLQGYHFTADSWEHIHFIGKIQGSDAGWTLGY |
| | | MLNLTNMIPAEQPLSTPLSHST |
| 33 | human CD39 variant | TQNKALPENVKYGIVLDAGSSHTSLYIYKWPAEKENDTGVVHQVE |
| | | ECRVKGPGISKFVQKVNEIGIYLTDCMERAMEVIPRSQHQETPVY |
| | | LGATAGMRLLRMESEELADRVLDVVERSLSNYPFDFQGARIITGQ |
| | | EEGAYGWITINYLLGKFSQKNQETFGALDLGGASTQVTFVPQNQT |
| | | IESPDNALQFRLYGKDYNVYTHSFLCYGKDQALWQKLAKDIQVAS |
| | | NEILRDPCFHPGYKKVVNVSDLYKTPCTKRFEMTLPFQQFEIQGI |
| | | GNYQQCHQSILELFNTSYCPYSQCAFNGIFLPPLQGDFGAFSAFY |
| | | FVMKFLNLTSEKVSQEKVTEMMKKFCAQPWEEIKTSYAGVKEKYL |
| | | SEFCFSGTYILSLLLQGYHFTADSWEHIHFIGKIQGSDAGWTLGY |
| | | MLNLTNMIPAEQPLSTPLSHST |
| 34 | human CD39 variant | TQNKALPENVKYGIVLDAGSSHTSLYIYKWPAEKENDTGVVHQVE |
| | | ECRVKGPGISKFVQKVNEIGIYLTDCMERAREVIPRSQHQETPVY |
| | | LGATAGMRLLRMESEELADRVLDVVERSLSNYPFDFQGARIITGQ |
| | | EEGAYGWITINYLLGKFSQKNQETFGALDLGGASTQVTFVPQNQT |
| | | IESPDNALQFRLYGKDYNVYTHSFLCYGKDQALWQKLAKDIQVAS |
| | | NEILRDPCFHPGYKKVVNVSDLYKTPCTKRFEMTLPFQQFEIQGI |
| | | GNYQQCHQSILELFNTSYCPYSQCAFNGIFLPPLQGDFGAFSAFY |
| | | FVMKFLNLTSEKVSQEKVTEMMKKFCAQPWEEIKTSYAGVKEKYL |
| | | SEFCFSGTYILSLLQQGYHFTADSWEHIHFIGKIQGSDAGWTLGY |
| | | MLNLTNMIPAEQPLSTPLSHST |
| 35 | human CD39 variant | TQNKALPENVKYGIVLDAGSSHTSLYIYKWPAEKENDTGVVHQVE |
| | | ECRVKGPGISKFVQKVNEIGIYLTDCMERAMEVIPRSQHQETPVY |
| | | LGATAGMRLLRMESEELADRVLDVVERSLSNYPFDFQGARIITGQ |
| | | EEGAYGWITINYLLGKFSQKNQETFGALDLGGASTQVTFVPQNQT |
| | | IESPDNALQFRLYGKDYNVYTHSFLCYGKDQALWQKLAKDIQVAS |
| | | NEILRDPCFHPGYKKVVNVSDLYKTPCTKRFEMTLPFQQFEIQGI |
| | | GNYQQCHQSILELFNTSYCPYSQCAFNGIFLPPLQGDFGAFSAFY |
| | | FVMKFLNLTSEKVSQEKVTEMMKKFCAQPWEEIKTSYAGVKEKYL |
| | | SEFCFSGTYILSLLQQGYHFTADSWEHIHFIGKIQGSDAGWTLGY |
| | | MLNLTNMIPAEQPLSTPLSHST |
| 36 | human CD39 variant | TQNKALPENVKYGIVLDAGSSHTSLYIYKWPAEKENDTGVVHQVE |
| | | ECRVKGPGISKFVQKVNEIGIYLTDCMERAMEVIPRSQHQETPVY |
| | | LGATAGMRLLRMESEELADRVLDVVERSLSNYPEDFQGARIITGQ |
| | | EEGAYGWITINYLLGKFSQKNQETFGALDLGGASTQVTFVPQNQT |
| | | IESPDNALQFRLYGKDYNVYTHSFLCYGKDQALWQKLAKDIQVAS |
| | | NEILRDPCFHPGYKKVVNVSDLYKTPCTKRFEMTLPFQQFEIQGI |
| | | GNYQQCHQSILELFNTSYCPYSQCAFNGIFLPPLQGDFGAFSAFY |
| | | SVMKFLNLTSEKVSQEKVTEMMKKFCAQPWEEIKTSYAGVKEKYL |
| | | SEYCFSGTYILSLLLQGYHFTADSWEHIHFIGKIQGSDAGWTLGY |
| | | MLNLTNMIPAEQPLSTPLSHST |
| 37 | human CD39 variant | APTSSSTQNKALPENVKYGIVLDAGSSHTSLYIYKWPAEKENDTG |
| | | VVHQVEECRVKGPGISKFVQKVNEIGIYLTDCMERAMEVIPRSQH |
| | | QETPVYLGATAGMRLLRMESEELADRVLDVVERSLSNYPFDFQGA |
| | | RIITGQEEGAYGWITINYLLGKFSQKNQETFGALDLGGASTQVTF |
| | | VPQNQTIESPDNALQFRLYGKDYNVYTHSFLCYGKDQALWQKLAK |
| | | DIQVASNEILRDPCFHPGYKKVVNVSDLYKTPCTKRFEMTLPFQQ |
| | | FEIQGIGNYQQCHQSILELFNTSYCPYSQCAFNGIFLPPLQGDFG |
| | | AFSAFYFVMKFLNLTSEKVSQEKVTEMMKKFCAQPWEEIKTSYAG |
| | | VKEKYLSEFCFSGTYILSLLLQGYHFTADSWEHIHFIGKIQGSDA |
| | | GWTLGYMLNLTNMIPAEQPLSTPLSHST |
| 38 | human CD39 variant | APTSSSTQNKALPENVKYGIVLDAGSSHTSLYIYKWPAEKENDTG |
| | | VVHQVEECRVKGPGISKFVQKVNEIGIYLTDCMERAREVIPRSQH |
| | | QETPVYLGATAGMRLLRMESEELADRVLDVVERSLSNYPFDFQGA |
| | | RIITGQEEGAYGWITINYLLGKFSQKNQETFGALDLGGASTQVTF |
| | | VPQNQTIESPDNALQFRLYGKDYNVYTHSFLCYGKDQALWQKLAK |
| | | DIQVASNEILRDPCFHPGYKKVVNVSDLYKTPCTKRFEMTLPFQQ |
| | | FEIQGIGNYQQCHQSILELFNTSYCPYSQCAFNGIFLPPLQGDFG |
| | | AFSAFYFVMKFLNLTSEKVSQEKVTEMMKKFCAQPWEEIKTSYAG |
| | | VKEKYLSEFCFSGTYILSLLQQGYHFTADSWEHIHFIGKIQGSDA |
| | | GWTLGYMLNLTNMIPAEQPLSTPLSHST |
| 39 | human CD39 variant | APTSSSTQNKALPENVKYGIVLDAGSSHTSLYIYKWPAEKENDTG |
| | | VVHQVEECRVKGPGISKFVQKVNEIGIYLTDCMERAMEVIPRSQH |
| | | QETPVYLGATAGMRLLRMESEELADRVLDVVERSLSNYPFDFQGA |
| | | RIITGQEEGAYGWITINYLLGKFSQKNQETFGALDLGGASTQVTF |
| | | VPQNQTIESPDNALQFRLYGKDYNVYTHSFLCYGKDQALWQKLAK |
| | | DIQVASNEILRDPCFHPGYKKVVNVSDLYKTPCTKRFEMTLPFQQ |
| | | FEIQGIGNYQQCHQSILELFNTSYCPYSQCAFNGIFLPPLQGDFG |
| | | AFSAFYFVMKFLNLTSEKVSQEKVTEMMKKFCAQPWEEIKTSYAG |
| | | VKEKYLSEFCFSGTYILSLLQQGYHFTADSWEHIHFIGKIQGSDA |
| | | GWTLGYMLNLTNMIPAEQPLSTPLSHST |
| 40 | human CD39 variant | APTTQNKALPENVKYGIVLDAGSSHTSLYIYKWPAEKENDTGVVH |
| | | QVEECRVKGPGISKFVQKVNEIGIYLTDCMERAREVIPRSQHQET |
| | | PVYLGATAGMRLLRMESEELADRVLDVVERSLSNYPFDFQGARII |
| | | TGQEEGAYGWITINYLLGKFSQKNQETFGALDLGGASTQVTFVPQ |
| | | NQTIESPDNALQFRLYGKDYNVYTHSFLCYGKDQALWQKLAKDIQ |
| | | VASNEILRDPCFHPGYKKVVNVSDLYKTPCTKRFEMTLPFQQFEI |
| | | QGIGNYQQCHQSILELFNTSYCPYSQCAFNGIFLPPLQGDFGAFS |
| | | AFYFVMKFLNLTSEKVSQEKVTEMMKKFCAQPWEEIKTSYAGVKE |
| | | KYLSEFCFSGTYILSLLQQGYHFTADSWEHIHFIGKIQGSDAGWT |
| | | LGYMLNLTNMIPAEQPLSTPLSHST |
| 41 | human CD39 variant | APTTQNKALPENVKYGIVLDAGSSHTSLYIYKWPAEKENDTGVVH |
| | | QVEECRVKGPGISKFVQKVNEIGIYLTDCMERAMEVIPRSQHQET |
| | | PVYLGATAGMRLLRMESEELADRVLDVVERSLSNYPFDFQGARII |
| | | TGQEEGAYGWITINYLLGKFSQKNQETFGALDLGGASTQVTFVPQ |
| | | NQTIESPDNALQFRLYGKDYNVYTHSFLCYGKDQALWQKLAKDIQ |
| | | VASNEILRDPCFHPGYKKVVNVSDLYKTPCTKRFEMTLPFQQFEI |
| | | QGIGNYQQCHQSILELFNTSYCPYSQCAFNGIFLPPLQGDFGAFS |
| | | AFYFVMKFLNLTSEKVSQEKVTEMMKKFCAQPWEEIKTSYAGVKE |
| | | KYLSEFCFSGTYILSLLQQGYHFTADSWEHIHFIGKIQGSDAGWT |
| | | LGYMLNLTNMIPAEQPLSTPLSHST |
| 42 | human CD39 variant | APTTQNKALPENVKYGIVLDAGSSHTSLYIYKWPAEKENDTGVVH |
| | | QVEECRVKGPGISKFVQKVNEIGIYLTDCMERAMEVIPRSQHQET |
| | | PVYLGATAGMRLLRMESEELADRVLDVVERSLSNYPFDFQGARII |
| | | TGQEEGAYGWITINYLLGKFSQKNQETFGALDLGGASTQVTFVPQ |
| | | NQTIESPDNALQFRLYGKDYNVYTHSFLCYGKDQALWQKLAKDIQ |
| | | VASNEILRDPCFHPGYKKVVNVSDLYKTPCTKRFEMTLPFQQFEI |
| | | QGIGNYQQCHQSILELFNTSYCPYSQCAFNGIFLPPLQGDFGAFS |
| | | AFYFVMKFLNLTSEKVSQEKVTEMMKKFCAQPWEEIKTSYAGVKE |
| | | KYLSEYCFSGTYILSLLLQGYHFTADSWEHIHFIGKIQGSDAGWT |
| | | LGYMLNLTNMIPAEQPLSTPLSHST |
| 43 | human CD39 variant | APTTQNKALPENVKYGIVLDAGSSHTSLYIYKWPAEKENDTGVVH |
| | | QVEECRVKGPGISKFVQKVNEIGIYLTDCMERAMEVIPRSQHQET |
| | | PVYLGATAGMRLLRMESEELADRVLDVVERSLSNYPFDFQGARII |
| | | TGQEEGAYGWITINYLLGKFSQKNQETFGALDLGGASTQVTFVPQ |
| | | NQTIESPDNALQFRLYGKDYNVYTHSFLCYGKDQALWQKLAKDIQ |
| | | VASNEILRDPCFHPGYKKVVNVSDLYKTPCTKRFEMTLPFQQFEI |
| | | QGIGNYQQCHQSILELFNTSYCPYSQCAFNGIFLPPLQGDFGAFS |
| | | AFYSVMKFLNLTSEKVSQEKVTEMMKKFCAQPWEEIKTSYAGVKE |
| | | KYLSEYCFSGTYILSLLLQGYHFTADSWEHIHFIGKIQGSDAGWT |
| | | LGYMLNLTNMIPAEQPLSTPLSHST |
| 44 | human CD39 variant | APTSSSTKKTQLTSSGTQNKALPENVKYGIVLDAGSSHTSLYIYK |
| | | WPAEKENDTGVVHQVEECRVKGPGISKFVQKVNEIGIYLTDCMER |
| | | AREVIPRSQHQETPVYLGATAGMRLLRMESEELADRVLDVVERSL |
| | | SNYPFDFQGARIITGQEEGAYGWITINYLLGKFSQKNQETFGALD |
| | | LGGASTQVTFVPQNQTIESPDNALQFRLYGKDYNVYTHSFLCYGK |
| | | DQALWQKLAKDIQVASNEILRDPCFHPGYKKVVNVSDLYKTPCTK |
| | | RFEMTLPFQQFEIQGIGNYQQCHQSILELFNTSYCPYSQCAFNGI |
| | | FLPPLQGDFGAFSAFYFVMKFLNLTSEKVSQEKVTEMMKKFCAQP |
| | | WEEIKTSYAGVKEKYLSEYCFSGTYILSLLLQGYHFTADSWEHIH |
| | | FIGKIQGSDAGWTLGYMLNLTNMIPAEQPLSTPLSHST |
| 45 | human CD39 variant | APTSSSTKKTQLTSSTQNKALPENVKYGIVLDAGSSHTSLYIYKW |
| | | PAEKENDTGVVHQVEECRVKGPGISKFVQKVNEIGIYLTDCMERA |
| | | MEVIPRSQHQETPVYLGATAGMRLLRMESEELADRVLDVVERSLS |
| | | NYPFDFQGARIITGQEEGAYGWITINYLLGKFSQKNQETFGALDL |
| | | GGASTQVTFVPQNQTIESPDNALQFRLYGKDYNVYTHSFLCYGKD |
| | | QALWQKLAKDIQVASNEILRDPCFHPGYKKVVNVSDLYKTPCTKR |
| | | FEMTLPFQQFEIQGIGNYQQCHQSILELFNTSYCPYSQCAFNGIF |
| | | LPPLQGDFGAFSAFYFVMKFLNLTSEKVSQEKVTEMMKKFCAQPW |
| | | EEIKTSYAGVNEKYLSEFCFSGTYILSLLLQGYHFTADSWEHIHF |
| | | IGKIQGSDAGWTLGYMLNLTNMIPAEQPLSTPLSHST |
| 46 | human CD39 variant | APTSSSTQNKALPENVKYGIVLDAGSSHTSLYIYKWPAEKENDTG |
| | | VVHQVEECRVKGPGISKFVQKVNEIGIYLTDCMERAMEVIPRSQH |
| | | QETPVYLGATAGMRLLRMESEELADRVLDVVERSLSNYPFDFQGA |
| | | RIITGQEEGAYGWITINYLLGKFSQKNQETFGALDLGGASTQVTF |
| | | VPQNQTIESPDNALQFRLYGKDYNVYTHSFLCYGKDQALWQKLAK |
| | | DIQVASNEILRDPCFHPGYKKVVNVSDLYKTPCTKRFEMTLPFQQ |
| | | FEIQGIGNYQQCHQSILELFNTSYCPYSQCAFNGIFLPPLQGDFG |
| | | AFSAFYSVMKFLNLTSEKVSQEKVTEMMKKFCAQPWEEIKTSYAG |
| | | VKEKYLSEYCFSGTYILSLLLQGYHFTADSWEHIHFIGKIQGSDA |
| | | GWTLGYMLNLTNMIPAEQPLSTPLSHST |
| 47 | human CD39 variant | APTTQNKALPENVKYGIVLDAGSSHTSLYIYKWPAEKENDTGVVH |
| | | QVEECRVKGPGISKFVQKVNEIGIYLTDCMERAMEVIPRSQHQET |
| | | PVYLGATAGMRLLRMESEELADRVLDVVERSLSNYPFDFQGARII |
| | | TGQEEGAYGWITINYLLGKFSQKNQETFGALDLGGASTQVTFVPQ |
| | | NQTIESPDNALQFRLYGKDYNVYTHSFLCYGKDQALWQKLAKDIQ |
| | | VASNEILRDPCFHPGYKKVVNVSDLYKTPCTKRFEMTLPFQQFEI |
| | | QGIGNYQQCHQSILELFNTSYCPYSQCAFNGIFLPPLQGDFGAFS |
| | | AFYFVMKFLNLTSEKVSQEKVTEMMKKFCAQPWEEIKTSYAGVKE |
| | | KYLSEFCFSGTYILSLLLQGYHFTADSWEHIHFIGKIQGSDAGWT |
| | | LGYMLNLTNMIPAEQPLSTPLSHST |
| 48 | human CD39 variant | APTTQNKALPENVKYGIVLDAGSSHTSLYIYKWPAEKENDTGVVH |
| | | QVEECRVKGPGISKFVQKVNEIGIYLTDCMERAREVIPRSQHQET |
| | | PVYLGATAGMRLLRMESEELADRVLDVVERSLSNYPFDFQGARII |
| | | TGQEEGAYGWITINYLLGKFSQKNQETFGALDLGGASTQVTFVPQ |
| | | NQTIESPDNALQFRLYGKDYNVYTHSFLCYGKDQALWQKLAKDIQ |
| | | VASNEILRDPCFHPGYKKVVNVSDLYKTPCTKRFEMTLPFQQFEI |
| | | QGIGNYQQCHQSILELFNTSYCPYSQCAFNGIFLPPLQGDFGAFS |
| | | AFYFVMKFLNLTSEKVSQEKVTEMMKKFCAQPWEEIKTSYAGVKE |
| | | KYLSEYCFSGTYILSLLLQGYHFTADSWEHIHFIGKIQGSDAGWT |
| | | LGYMLNLTNMIPAEQPLSTPLSHST |
| 49 | human CD39 variant | APTTQNKALPENVKYGIVLDAGSSHTSLYIYKWPAEKENDTGVVH |
| | | QVEECRVKGPGISKEVQKVNEIGIYLTDCMERAREVIPRSQHQET |
| | | PVYLGATAGMRLLRMESEELADRVLDVVERSLSNYPFDFQGARII |
| | | TGQEEGAYGWITINYLLGKFSQKNQETFGALDLGGASTQVTFVPQ |
| | | NQTIESPDNALQFRLYGKDYNVYTHSFLCYGKDQALWQKLAKDIQ |
| | | VASNEILRDPCFHPGYKKVVNVSDLYKTPCTKRFEMTLPFQQFEI |
| | | QGIGNYQQCHQSILELFNTSYCPYSQCAFNGIFLPPLQGDFGAFS |
| | | AFYSVMKFLNLTSEKVSQEKVTEMMKKFCAQPWEEIKTSYAGVKE |
| | | KYLSEYCFSGTYILSLLLQGYHFTADSWEHIHFIGKIQGSDAGWT |
| | | LGYMLNLTNMIPAEQPLSTPLSHST |
| 50 | human CD39 | MEDTKESNVKTFCSKNILAILGESSIIAVIALLAVGLTQNKALPE |
| | | NVKYGIVLDAGSSHTSLYIYKWPAEKENDTGVVHQVEECRVKGPG |
| | | ISKFVQKVNEIGIYLTDCMERAREVIPRSQHQETPVYLGATAGMR |
| | | LLRMESEELADRVLDVVERSLSNYPFDFQGARIITGQEEGAYGWI |
| | | TINYLLGKFSQKTRWFSIVPYETNNQETFGALDLGGASTQVTFVP |
| | | QNQTIESPDNALQFRLYGKDYNVYTHSFLCYGKDQALWQKLAKDI |
| | | QVASNEILRDPCFHPGYKKVVNVSDLYKTPCTKRFEMTLPFQQFE |
| | | IQGIGNYQQCHQSILELFNTSYCPYSQCAFNGIFLPPLQGDFGAF |
| | | SAFYFVMKFLNLTSEKVSQEKVTEMMKKFCAQPWEEIKTSYAGVK |
| | | EKYLSEYCFSGTYILSLLLQGYHFTADSWEHIHFIGKIQGSDAGW |
| | | TLGYMLNLTNMIPAEQPLSTPLSHSTYVFLMVLFSLVLFTVAIIG |
| | | LLIFHKPSYFWKDMV |
| 51 | gRNA CCL2-01 | TCTTGCGGAGAACTGCATGGTGG |
| 52 | gRNA CCL2-02 | ATCTATGTGCTGGCCCAGCCAGG |
| 53 | gRNA CCL2-03 | GGAGAATAAACCAGAAAGGGGGG |
| 54 | gRNA CCL2-04 | GCTACTCATTTACCGCCAAGAGG |

1. A method of producing CD39 or a variant thereof, comprising the steps of
(a) providing CHO cells capable of producing CD39 or a variant thereof;
(b) cultivating the CHO cells in a cell culture under conditions which allow for the production of CD39 or the variant thereof;
(c) obtaining CD39 or the variant thereof from the cell culture; and
(d) optionally processing CD39 or the variant thereof;
wherein the CHO cells are engineered to reduce the production of CCL2 in the CHO cells.

2. The method according to claim 1, wherein production of CCL2 in the CHO cells is reduced by knockdown or knockout of CCL2 expression or degradation of CCL2 protein.

3. The method according to claim 1 or 2, wherein the CHO cells are engineered to produce a miRNA targeting the endogenous CCL2 of the CHO cells, wherein the miRNA targeting CCL2 preferably has a nucleotide sequence selected from the group consisting of SEQ ID NOs: 26 and 28.

4. The method according to claim 3, wherein the CHO cells comprise an expression cassette for production of the miRNA targeting CCL2, wherein the expression cassette comprises an intronic sequence comprising a template sequence for a pri-miRNA, wherein the pri-miRNA is suitable to be processed in the CHO cell to form the miRNA targeting CCL2.

5. The method according to claim 4, wherein the pri-miRNA comprises, from 5′ to 3′,
(i) a 5′ miRNA scaffold stem, optionally comprising the nucleotide sequence of SEQ ID NO: 1-4,
(ii) a passenger strand, having a nucleotide sequence complementary to the sequence of the miRNA, optionally comprising one or two mismatches,
(iii) a miRNA scaffold loop, optionally comprising the nucleotide sequence of SEQ ID NO: 8,
(iv) a guide strand, having the nucleotide sequence of the miRNA, and
(v) a 3′ miRNA scaffold stem, optionally comprising the nucleotide sequence of SEQ ID NO: 11-14;
wherein the positions of the passenger strand and guide strand may be switched, and wherein optionally the passenger strand has the nucleotide sequence of SEQ ID NO: 25 and the guide strand has the nucleotide sequence of SEQ ID NO: 26, or the passenger strand has the nucleotide sequence of SEQ ID NO: 27 and the guide strand has the nucleotide sequence of SEQ ID NO: 28.

6. The method according to any one of claims 1 to 5, wherein the method is for producing a variant of CD39 which has one or more of the following characteristics:
(i) it is a soluble variant lacking the membrane anchors of human CD39;
(ii) it has N terminal, C terminal and central deletions compared to human CD39;
(iii) it has an N terminal deletion of 30 to 50 amino acids, a C terminal deletion of 20 to 40 amino acids, and a central deletion of 10 to 15 amino acids compared to human CD39;
(iv) it comprise up to 5 point mutations compared to human CD39;
(v) it has an amino acid sequence selected from the group consisting of SEQ ID NOs: 29-49.

7. The method according to any one of claims 1 to 6, wherein CD39 or the variant thereof obtained after step (c) is in a composition which comprises CCL2 in a concentration of 100 ppm or lower, preferably 25 ppm or lower, more preferably 10 ppm or lower, and/or which comprises CCL2 in a concentration which is at least 10-times lower, preferably 25-times lower, more preferably 100-times lower, compared to a composition obtained with the same method after step (c) using CHO cells which are not engineered to reduce the production of CCL2 in the CHO cells and/or wherein CD39 or the variant thereof obtained after step (d) is in a composition which comprises CCL2 in a concentration of 25 ppm or lower, preferably 10 ppm or lower, more preferably 5 ppm or lower, and/or which comprises CCL2 in a concentration which is at least 10-times lower, preferably 25-times lower, more preferably 100-times lower, compared to a composition obtained with the same method after step (d) using CHO cells which are not engineered to reduce the production of CCL2 in the CHO cells.

8. The method according to any one of claims 1 to 7, wherein step (c) comprises
(c1) separating the CHO cells from cell culture supernatant containing CD39 or the variant thereof;
(c2) separating CD39 or the variant thereof from the cell culture supernatant using anion exchange chromatography;
(c3) further purifying CD39 or the variant thereof using hydrophobic interaction chromatography; and
(c4) further purifying CD39 or the variant thereof using anion exchange chromatography.

9. The method according to claim 8, wherein CD39 or the variant thereof obtained after step (c1) is in a composition which comprises CCL2 in a concentration of 100 ppm or lower, preferably 25 ppm or lower, more preferably 10 ppm or lower, and/or which comprises CCL2 in a concentration which is at least 10-times lower, preferably 25-times lower, more preferably 100-times lower, compared to a composition obtained with the same method after step (c1) using CHO cells which are not engineered to reduce the production of CCL2 in the CHO cells; and/or wherein CD39 or the variant thereof obtained after step (c4) is in a composition which comprises CCL2 in a concentration of 25 ppm or lower, preferably 10 ppm or lower, more preferably 5 ppm or lower, and/or which comprises CCL2 in a concentration which is at least 10-times lower, preferably 25-times lower, more preferably 100-times lower, compared to a composition obtained with the same method after step (c4) using CHO cells which are not engineered to reduce the production of CCL2 in the CHO cells.

10. The method according to any one of claims 1 to 9, wherein step (d) comprises providing a pharmaceutical formulation comprising CD39 or the variant thereof.

11. A CHO cell which is capable of producing CD39 or a variant thereof and which is engineered to reduce the production of CCL2 in the CHO cell.

12. The CHO cell according to claim 11, wherein the CHO cell is engineered to reduce the production of CCL2 in the CHO cell as defined in any one of claims 2 to 7.

13. A method of improving production of CD39 or a variant thereof in a CHO cell, comprising the steps of
(a-i) providing a CHO cell capable of producing CD39 or a variant thereof;
(a-ii) engineering the CHO cell so as to reduce the production of CCL2 in the CHO cell.

14. The method according to claim 13, wherein the CHO cell is engineered in step (a-ii) as defined in any one of claims 2 to 7.

15. The method according to claim 13 or 14, wherein step (a-ii) includes introducing a vector nucleic acid into the CHO cell, wherein the vector nucleic acid comprises an expression cassette for expression of a miRNA targeting CCL2.

16. The method according to any one of claims 13 to 15, wherein engineering the CHO cell reduces the level of CCL2 mRNA and/or the level of CCL2 protein in the CHO cell by at least 5-fold, preferably at least 10-fold, more preferably at least 25-fold, compared to the same CHO cell prior to step (a-ii).

17. The method according to any one of claims 13 to 16, wherein the method further comprises the steps of
(b) cultivating the CHO cell obtained in step (a-ii) in a cell culture under conditions which allow for proliferation of the CHO cell and simultaneous and/or subsequent production of CD39 or the variant thereof;
(c) obtaining said CD39 or the variant thereof from the cell culture; and
(d) optionally processing CD39 or the variant thereof.

18. The method according to claim 17, wherein step (c) comprises
(c1) separating the CHO cells from cell culture supernatant containing CD39 or the variant thereof;
(c2) separating CD39 or the variant thereof from the cell culture supernatant using anion exchange chromatography;
(c3) further purifying CD39 or the variant thereof using hydrophobic interaction chromatography; and
(c4) further purifying CD39 or the variant thereof using anion exchange chromatography;
and/or
wherein step (d) comprises providing a pharmaceutical formulation comprising CD39 or the variant thereof.

19. An expression cassette for expression of a miRNA in a CHO cell, comprising a template sequence for a pri-miRNA, wherein the pri-miRNA is suitable to be processed in a CHO cell to form a miRNA targeting CCL2.

20. The expression cassette according to claim 19, having the features as defined in any one of claims 3 to 5.

21. A vector nucleic acid for transfection of a CHO cell, comprising the expression cassette according to claim 19 or 20.

22. A method for producing a CHO cell which is capable of expressing CD39 or a variant thereof, comprising introducing a vector nucleic acid according to claim 21 into a CHO cell.

23. A composition comprising CD39 or a variant thereof and CCL2, wherein the composition is obtained by production using CHO cells according to claim 11 or 12, and wherein
(i) the amount of CCL2 in the composition is at least 10-times lower compared to the same composition obtained by production using CHO cells which were not engineered to reduce the production of CCL2 in the CHO cells; and/or
(ii) the composition comprises CCL2 in a concentration of 100 ppm or lower, preferably 25 ppm or lower, more preferably 10 ppm or lower.

24. A CHO cell which is engineered to reduce the production of CCL2 in the CHO cell, especially as defined in any one of claims 2 to 7.
